(19) United States
(12) Patent Application Publication
(10) Pub. No.: US 2025/0364121 A1
(43) Pub. Date: Nov. 27, 2025

(54) GLOBAL AND LOCAL SEARCH-BASED CLASSIFICATION OF TEXT

(71) Applicant: GE Precision Healthcare LLC, Waukesha, WI (US)

(72) Inventors: Seyed Iman Zare Estakhraji, Redwood City, CA (US); R. Marc Lebel, Calgary (CA); Allison Li, Milwaukee, WI (US); Arish Ali, Hyderabad (IN); Grace Ellen Gibson, Milwaukee, WI (US)

(21) Appl. No.: 18/670,881

(22) Filed: May 22, 2024

(51) Int. Cl. G16H 40/20; G16H 50/70

(52) U.S. Cl. CPC G16H 40/20; G16H 50/70

(57) ABSTRACT

Systems or techniques that facilitate global and local search-based classification of text are provided. In various embodiments, a system can access a new medical order associated with a medical patient. In various aspects, the system can compute: one or more global vector representations of the new medical order; and one or more local vector representations for respective ones or combinations of a set of textual sections that make up the new medical order, thereby yielding a set of local vector representations of the new medical order. In various instances, the system can identify a new classification label for the new medical order, based on searching an historical order-label database using both the set of global vector representations and the set of local vector representations.

### TECHNICAL FIELD

The subject disclosure relates generally to text classification, and more specifically to global and local search-based classification of text.

### BACKGROUND

When given a medical order of a medical patient, it can be desired to classify that medical order to determine follow-on medical activity for the medical patient. Existing techniques facilitate such classification using machine learning. Unfortunately, such existing techniques are excessively computationally and regulatorily expensive.

Accordingly, systems or techniques that can address one or more of these technical problems can be desirable.

### SUMMARY

The following presents a summary to provide a basic understanding of one or more embodiments. This summary is not intended to identify key or critical elements, or delineate any scope of the particular embodiments or any scope of the claims. Its sole purpose is to present concepts in a simplified form as a prelude to the more detailed description that is presented later. In one or more embodiments described herein, devices, systems, computer-implemented methods, apparatus or computer program products that facilitate global and local search-based classification of text are described.

According to one or more embodiments, a system is provided. The system can comprise a non-transitory computer-readable memory that can store computer-executable components. The system can further comprise a processor that can be operably coupled to the non-transitory computer-readable memory and that can execute the computer-executable components stored in the non-transitory computer-readable memory. In various embodiments, the computer-executable components can comprise an access component that can access a new medical order associated with a medical patient. In various aspects, the computer-executable components can comprise a vector component that can compute: one or more global vector representations of the new medical order; and one or more local vector representations for respective ones or combinations of a set of textual sections that make up the new medical order, thereby yielding a set of local vector representations of the new medical order. In various instances, the computer-executable components can comprise a search component that can identify a new classification label for the new medical order, based on searching an historical order-label database using both the set of global vector representations and the set of local vector representations.

According to one or more embodiments, a computer-implemented method is provided. In various embodiments, the computer-implemented method can comprise accessing, by a device operatively coupled to a processor, a new medical order associated with a medical patient. In various aspects, the computer-implemented method can comprise computing, by the device: one or more global vector representations of the new medical order; and one or more local vector representations for respective ones or combinations of a set of textual sections that make up the new medical order, thereby yielding a set of local vector representations of the new medical order. In various instances, the computer-implemented method can comprise identifying, by the device, a new classification label for the new medical order, based on searching an historical order-label database using both the set of global vector representations and the set of local vector representations.

According to one or more embodiments, a computer program product for facilitating global and local search-based classification of text is provided. In various embodiments, the computer program product can comprise a non-transitory computer-readable memory having program instructions embodied therewith. In various aspects, the program instructions can be executable by a processor to cause the processor to access a new textual document. In various instances, the program instructions can be further executable to cause the processor to compute: one or more global vector representations of the new textual document; and one or more local vector representations for respective ones or combinations of a set of sections of the new textual document, thereby yielding a set of local vector representations of the new textual document. In various cases, the program instructions can be further executable to cause the processor to identify a new classification label for the new textual document, based on searching an historical document-label database using both the set of global vector representations and the set of local vector representations.

### DESCRIPTION OF THE DRAWINGS

FIG. 1 illustrates a block diagram of an example, non-limiting system that facilitates global and local search-based classification of text in accordance with one or more embodiments described herein.

FIG. 2 illustrates a block diagram of an example, non-limiting system including global vectors and local vectors that facilitates global and local search-based classification of text in accordance with one or more embodiments described herein.

FIG. 3 illustrates an example, non-limiting block diagram showing global vectors and local vectors in accordance with one or more embodiments described herein.

FIG. 4 illustrates a block diagram of an example, non-limiting system including an historical order-label database and a new classification label that facilitates global and local search-based classification of text in accordance with one or more embodiments described herein.

FIGS. 5-9 illustrate example, non-limiting block diagrams showing how a new classification label can be identified by searching an historical order-label database with global and local vectors in accordance with one or more embodiments described herein.

FIG. 10 illustrates an example, non-limiting block diagram showing how a new entry can be inserted into an historical order-label database in accordance with one or more embodiments described herein.

FIG. 11 illustrates a block diagram of an example, non-limiting system including a device instruction that facilitates global and local search-based classification of text in accordance with one or more embodiments described herein.

FIG. 12 illustrates a flow diagram of an example, non-limiting computer-implemented method that facilitates global and local search-based classification of text in accordance with one or more embodiments described herein.

FIG. 13 illustrates a block diagram of an example, non-limiting operating environment in which one or more embodiments described herein can be facilitated.

FIG. 14 illustrates an example networking environment operable to execute various implementations described herein.

### DETAILED DESCRIPTION

The following detailed description is merely illustrative and is not intended to limit embodiments or application/uses of embodiments. Furthermore, there is no intention to be bound by any expressed or implied information presented in the preceding Background or Summary sections, or in the Detailed Description section.

One or more embodiments are now described with reference to the drawings, wherein like referenced numerals are used to refer to like elements throughout. In the following description, for purposes of explanation, numerous specific details are set forth in order to provide a more thorough understanding of the one or more embodiments. It is evident, however, in various cases, that the one or more embodiments can be practiced without these specific details.

When given a medical order of a medical patient (e.g., human, animal, or otherwise), it can be desired to classify that medical order, so as to determine follow-on medical activity for the medical patient. Indeed, the medical order can be an electronic textual document having multiple sections, segments, or text fields (e.g., ordering department, patient demographics, patient medical history), that is written or typed by a medical professional who attends to the medical patient, and that describes, requests, or otherwise calls for some medical action (e.g., a specified imaging protocol, a specified surgical intervention) to be performed on or implemented with respect to the medical patient. So, a plurality of categories or classes can be defined, where each category or class can represent a respective medical action that can possibly be performed on or implemented with respect to the medical patient, and the specific medical action that has been actually requested or prescribed for the medical patient can be automatically identified by electronically classifying the medical order into one of that plurality of categories or classes.

Existing techniques facilitate such automated classification via machine learning. In particular, some existing techniques use specially trained machine learning classifiers to facilitate such medical order classification. In particular, such existing techniques involve training a machine learning classifier (e.g., deep learning neural network) from scratch using training medical orders that are each known or deemed to correspond to a respective ground-truth classification label. Accordingly, after training, the machine learning classifier can receive any given medical order as input and can produce as output a predicted or inferred classification label for that inputted medical order. Other existing techniques use fine-tuned large language models (LLMs) to facilitate medical order classification. In particular, for any given medical order, such other existing techniques involve concatenating that given medical order with a textual prompt that asks what medical action, activity, or prescription is being requested or called for by the given medical order. Such other existing techniques then involve feeding that concatenation as input to an LLM, which causes the LLM to produce a synthesized textual response that answers the prompt: that is, that identifies what specific medical action, activity, or prescription is (as inferred or predicted by the LLM) being requested or called for by the given medical order. So, the synthesized textual response can be considered as indicating the class to which the given medical order is inferred or predicted to belong. However, because LLMs (e.g., ChatGPT) are highly generalized machine learning models, they are not often exposed to medical orders or questions regarding medical orders. Accordingly, it has been found that LLMs classify medical orders sufficiently accurately only after being fine-tuned (e.g., re-trained without internal parameter reinitialization) to handle medical orders. In other words, in the absence of fine-tuning, it has been found that LLMs very often incorrectly identify the specific medical actions, activities, or prescriptions that are called for by inputted medical orders.

Unfortunately, existing techniques are disadvantageous for various reasons.

First, the computational footprint of existing techniques can be massive. Indeed, existing techniques require either specially trained machine learning classifiers or fine-tuned LLMs. A machine learning classifier can have hundreds of thousands or even millions of trainable internal parameters (e.g., convolutional kernels, weight matrices, bias values). Moreover, the number of trainable internal parameters of LLMs can stretch into the billions or even trillions (e.g., ChatGPT has hundreds of billions of internal parameters). Indeed, LLMs can be so computationally expensive that existing techniques actively avoid LLM ensembling, despite the fact that ensembling would be theoretically beneficially since different LLMs (e.g., ChatGPT, Claude®, Bard®), which are initially trained on different datasets, can be considered as learning to focus on or otherwise pay attention to different aspects of semantic content. Because machine learning classifiers and LLMs can be so large, commensurately significant amounts of computer memory and processing capacity can be needed to implement them. Accordingly, a cloud server that offers medical order classification as a service according to such existing techniques must shoulder the burden of maintaining such significant amounts of computer memory and processing capacity.

Second, the already-significant computational footprint of existing techniques is exacerbated by the wide variety of medical orders across different medical sites. Indeed, different medical sites (e.g., different hospitals) can serve different populations of patients (e.g., geriatric patients, pediatric patients, pregnant patients) in different geographic locations (e.g., different cities, states, or countries) using different types of medical actions, activities, or prescriptions (e.g., the prescriptions ordered for geriatric patients can be different from those ordered for pediatric patients; the prescriptions ordered for pregnant patients at a first medical site can be different from those ordered for pregnant patients at a second medical site). Accordingly, medical order classifications can differ widely across different medical sites. Furthermore, different medical sites can have their own unique or idiosyncratic jargon or phraseology. Thus, even if two different medical orders actually call for or request the same prescription as each other, they might do so using significantly different language (e.g., using significantly differently-worded terms or phrases). It has been found that a single, unified machine learning classifier or LLM is, even after exhaustive training, unable to sufficiently accurately or reliably handle such wide medical order variety (e.g., the single classifier or LLM becomes a jack of all trades and master of none with respect to the different medical sites). So, to handle all of this medical order variety, existing techniques implement a distinct or separate machine learning classifier or LLM that is specially trained or fine-tuned on the specific medical orders of each distinct or separate medical site. Accordingly, the internal parameters of each distinct or separate classifier or LLM can be considered as being uniquely updated or optimized so as to accurately or reliably classify the idiosyncratic medical orders of a respective medical site. Since the computational cost (e.g., in terms of computer memory and processing capacity) of maintaining one classifier or LLM can be already massive, the computational cost of maintaining a multitude of such classifiers or LLMs (e.g., respectively corresponding to a multitude of medical sites) can be gargantuan. Again, a cloud server that offers medical order classification as a service according to such existing techniques must therefore shoulder that gargantuan burden of maintaining sufficient amounts of computer memory and processing capacity.

Third, existing techniques require continual learning. In particular, the medical field can be considered as an operational environment that is highly vulnerable to data distribution drift. After all, the types of patients served by, the types of medical professionals employed by, and the types of medical equipment or treatments prescribed by a given medical site can gradually or rapidly change, evolve, or otherwise shift over time. Accordingly, the patterns, distributions, internal signatures, or other statistical metrics of medical orders that originate from that given medical site can likewise change, evolve, or otherwise shift over time. So, a machine learning classifier or LLM that has been specially trained or fine-tuned to classify medical orders for the given medical site can do so accurately or reliably initially after being trained, but that machine learning classifier or LLM can become progressively less likely to do so accurately or reliably as the characteristics of medical orders from the given medical site change or drift over time. To address this, the machine learning classifier or LLM can be periodically or frequently re-trained on new training medical orders from the given medical site. Although such re-training can enable the machine learning classifier or LLM to maintain medical order classification accuracy or confidence, such re-training can be associated with significant disadvantages. Indeed, re-training can be quite time-consuming (e.g., requiring tens of hours depending upon the number of internal model parameters that need to be updated) and often requires the collation of voluminous amounts of annotated training data (e.g., which can entail significant manual effort for whichever technicians are tasked with implementing the re-training). Such detriments of re-training can be reduced by using less training data, but doing so simultaneously reduces the efficacy of such retraining (e.g., the less re-training that is performed, the more vulnerable a model is to data drift). Furthermore, a machine learning model that is re-trained can be vulnerable to catastrophic forgetting (e.g., to abruptly and drastically forgetting previously learned information upon learning new information). Further still, recent regulatory restrictions have been enacted by various governmental entities, which require machine learning models that are implemented in the medical field to undergo rigorous certification or verification approval processes before medical professionals are permitted to trust or rely upon their inferences or predictions. So, a machine learning classifier or LLM that has already undergone such intensive approval processes can, each time it is re-trained, be required to undergo such intensive approval processes all over again (e.g., since re-training involves changing the learnable weights of the machine learning model or LLM).

Accordingly, systems or techniques that can address one or more of these technical problems can be desirable.

Various embodiments described herein can address one or more of these technical problems. One or more embodiments described herein can include systems, computer-implemented methods, apparatus, or computer program products that can facilitate global and local search-based classification of text. In particular, the inventors of various embodiments described herein devised various techniques that can facilitate accurate or reliable classification of medical orders, without suffering from various disadvantages that plague existing techniques.

More specifically, various embodiments described herein can involve generating, for any given medical order, a global vector representation (e.g., an embedding or latent vector corresponding to the entirety of the given medical order) and various local vector representations (e.g., embeddings or latent vectors corresponding to respective sub-portions or sub-parts of the given medical order). In some cases, such global and local vectors can be computed using non-machine-learning vectorization techniques. In other cases, such global and local vectors can be computed by any suitable pre-trained encoder (e.g., a text-to-vector encoder from any already-trained medical order classifier; a text-to-vector encoder from any already-trained LLM; a text-to-vector encoder from any other suitable already-trained machine learning model, even if such model is completely unrelated to medical orders). As described herein, there can be an order-label database that stores: past medical orders; the classification labels that were known or selected for those past medical orders; and the global and local vector representations that were computed for those past medical orders. Accordingly, various embodiments described herein can involve searching the order-label database using the global and local vector representations, so as to identify a past medical order that is most semantically similar to the given medical order. Thus, whatever classification label was selected or chosen for that most-similar past medical order can be automatically selected or chosen for the given medical order. In this way, the given medical order can be classified.

Note that various embodiments described herein can avoid various of the aforementioned pitfalls experienced by existing techniques.

Indeed, the computational footprint of various embodiments described herein can be much smaller than that of existing techniques. As mentioned above, existing techniques rely on specially trained machine learning classifiers or fine-tuned LLMs, which can contain vast amounts of internal parameters (e.g., millions, billions, or even trillions), and which can thus consume commensurately vast amounts of computer memory or processing power (e.g., the learned parameters of ChatGPT take up thousands of gigabytes of computer memory). In stark contrast, various embodiments described herein can consume orders of magnitude fewer computing resources. Indeed, the brunt of the computational footprint of various embodiments described herein can come from two sources: the order-label database; and the vectorization technique. The present inventors experimentally verified that an order-label database comprising merely a few thousand (or even as low as a few hundred) past medical orders can cause various embodiments described herein to achieve comparable (in some cases, better) classification performance than existing techniques. Such an order-label database can clearly consume significantly less computer memory than specially trained machine learning classifiers or fine-tuned LLMs. Regarding vectorization techniques, some embodiments described herein can utilize non-machine-learning vectorizers to convert medical orders into global and local vectors. Such non-machine-learning vectorizers can lack learnable or trainable internal parameters and thus consume negligible computing resources in comparison with specially trained machine learning classifiers or fine-tuned LLMs. Now, other embodiments described herein can utilize pre-trained encoders to convert medical orders into global and local vectors. A pre-trained encoder can come from (e.g., can be an upstream portion of) a specially trained machine learning classifier, a fine-tuned LLM, or any other suitable text-analysis machine learning model. Thus, although a pre-trained encoder can have a non-zero number of learnable or trainable internal parameters, it can have merely a fraction of the total number of parameters utilized by existing techniques. For at least these reasons, various embodiments described herein can be considered as being significantly lighter or less computationally intensive than existing techniques.

Next, when given multiple different medical sites for which medical order classification is desired, there is no need to uniquely tailor a distinct instantiation of various embodiments described herein to each of such multiple medical sites. As mentioned above, existing techniques achieve satisfactory classification accuracy by training a separate or distinct classifier or LLM for each distinct medical site, which can significantly exacerbate the already-significant computational footprint of existing techniques. For example, suppose that it is desired to provide medical order classification services to three different medical sites: hospital A; hospital B; and hospital C. In such case, existing techniques would: train or fine-tune a first classifier or LLM (e.g., a first version of ChatGPT) to perform medical order classification specifically for the hospital A; train or fine-tune a second classifier or LLM (e.g., a second version of ChatGPT) to perform medical order classification specifically for the hospital B; and train or fine-tune a third classifier or LLM (e.g., a third version of ChatGPT) to perform medical order classification specifically for the hospital C. In stark contrast, various embodiments described herein do not require such multiplicity. To continue the above example, various embodiments described herein do not require (although, they do permit) implementing: a first order-label database and first vectorization technique for the hospital A; a second order-label database and second vectorization technique for the hospital B; and a third order-label database and third vectorization technique for the hospital C. Instead, various embodiments described herein can utilize a single, centralized order-label database and a single, centralized vectorization technique for all of hospitals A, B, and C. In other cases, various embodiments described herein can utilize separate order-label databases but a single, centralized vectorization technique for all of hospitals A, B, and C.

This benefit (e.g., no need for distinct instantiation for each medical site) can be due to the implementation of both global and local vector representations of medical orders. Indeed, the present inventors realized that there can be an inconsistency or mismatch between: the global vector representation of an entire medical order; and the aggregation of local vector representations of respective parts of the medical order. For example, suppose that a medical order is made up of x different textual sections, for any suitable positive integer x>1. In such case, a global vector can be computed for the entirety of that medical order, and a total of x local vector representations can be computed for that medical order (e.g., one local vector per textual section). The present inventors recognized that the average of those x local vectors is often not equal to the global vector. In other words, there can be a global-local disconnect for vector representations of medical orders. The present inventors realized that such global-local disconnect can mean that vectorization techniques can capture different types of semantic information depending upon the level of textual granularity at which such vectorization techniques are implemented. Accordingly, leveraging both global and local vectors to search for semantically similar medical orders can be considered as a robust search strategy that deepens, enhances, or otherwise enriches the semantic search space. In other words, global and local vectors can be considered as not substitutes for each other; instead, global and local vectors can be considered as complementing each other, so as to more fully or more completely represent the semantic meaning of any given medical order. Such fuller or more complete capture of semantic meaning can be better able to handle or discern the wide semantic variety arising from medical orders of multiple different medical sites, as compared to existing techniques.

Moreover, various embodiments described herein can involve no re-training or continual learning. As mentioned above, existing techniques require specially trained classifiers or fine-tuned LLMs to be periodically or continually re-trained or re-fine-tuned, so as to deal with the problem of data distribution drift. In stark contrast, various embodiments described herein can eschew such re-training or re-fine-tuning. Indeed, some embodiments described herein can utilize non-machine-learning vectorization techniques. In such embodiments, there is nothing that can be re-trained or re-fine-tuned (e.g., such embodiments contain no learned or trainable parameters that can be incrementally updated during re-training). Furthermore, although other embodiments described herein can utilize pre-trained encoders to vectorize medical orders, such pre-trained encoders can be accurately or reliably operated without re-training or re-fine-tuning. After all, various embodiments described herein can operate by searching the order-label database for past medical orders that are semantically similar (e.g., in terms of global and local vector representations) to a given medical order. So, any sudden or dramatic drifts in medical orders at any medical site can be easily taken into account by inserting a small number of drifted exemplars (e.g., a dozen or fewer drifted medical orders and their corresponding classification labels) into the order-label database. Contrast this nearly-effortless insertion with re-training, which instead requires the effort-intensive collation of voluminous annotated training data (e.g., thousands of drifted medical orders and their corresponding classification labels), and which also requires significant amounts of computation time (e.g., backpropagation of LLMs can consume tens of hours, depending on the number of training batches or epochs). Furthermore, because various embodiments described herein can involve no re-training, the problems of regulatory re-compliance and catastrophic forgetting can be avoided (e.g., various embodiments described here cannot “forget” since re-training is omitted; various embodiments described herein need not be repetitively re-certified to be in compliance with Food and Drug Administration regulations).

Various embodiments described herein can be considered as a computerized tool (e.g., any suitable combination of computer-executable hardware or computer-executable software) that can facilitate global and local search-based classification of text. In various aspects, such computerized tool can comprise an access component, a vector component, a search component, or an execution component.

In various embodiments, there can be a medical device that monitors or is otherwise clinically associated with a medical patient. In various aspects, the medical device can be any suitable type of medical image-capture equipment or modality (e.g., a computed tomography (CT) scanner, a magnetic resonance imaging (MRI) scanner, an X-ray scanner, an ultrasound scanner, a positron emission tomography (PET) scanner, a nuclear medicine (NM) scanner). In various instances, the medical device can instead be any suitable type of automated medication dispenser (e.g., an intravenous infusion pump, a respirator, a hemodialysis machine, an aerosol tent or mask, a nebulizer). In various aspects, the medical device can instead be any suitable type of automated surgical equipment or modality (e.g., robotically-assisted surgery machine for laparoscopic procedures).

In various embodiments, there can be a new medical order that is associated with the medical patient. In various aspects, the new medical order can be any suitable electronic textual document that: describes or explains any suitable demographic information about the medical patient; describes or explains any suitable medical observations about the medical patient; and requests or prescribes some medical action to be taken for or with respect to the medical patient. In some cases, the requested or prescribed medical action can be any suitable automated task that is performable by the medical device (e.g., automated imaging protocol, automated medication dispensation, automated surgical intervention). In various aspects, the new medical order can be electronically typed or written into any suitable computerized device by a medical professional who is attending to the medical patient.

In various instances, it can be desired to automatically classify the new medical order so as to determine what medical action it requests or prescribes. As described herein, the computerized tool can accomplish such classification.

In various embodiments, the access component of the computerized tool can electronically access the new medical order. For instance, the access component can receive, retrieve, or otherwise obtain the new medical order from any suitable centralized or decentralized data structures (e.g., graph data structures, relational data structures, hybrid data structures). Likewise, the access component can electronically access the medical device. For instance, the access component can electronically interface or communicate with (e.g., send electronic commands to, read electronic signals from) the medical device. In any case, the access component can be considered as a conduit through which other components of the computerized tool can electronically interact with (e.g., read, write, edit, copy, manipulate, execute, activate, deactivate, modify) the new medical order or the medical device.

In various embodiments, the vector component of the computerized tool can electronically generate a global vector and a set of local vectors, by applying any suitable vectorizer to the new medical order. More specifically, the vectorizer can be any suitable text-to-vector transformation technique (e.g., any suitable algorithm that can convert any given piece of text into a numerical representation). In some cases, the vectorizer can be any suitable non-machine-learning vectorizer, such as a term-frequency-inverse-domain-frequency (TF-IDF) vectorization technique. In other cases, the vectorizer can instead be any suitable machine learning vectorizer, such as an encoder from any suitable pre-trained text-analysis machine learning model (e.g., the encoder from an already-trained variational autoencoder; the encoder from an already-trained text classifier; the encoder from an already-trained LLM). Note that such other cases can be considered as repurposing, recycling, or borrowing the encoder of that pre-trained text-analysis machine learning model.

In various aspects, the global vector can, despite its name, be any suitable mathematical quantity (e.g., one or more scalars, one or more vectors, one or more matrices, one or more tensors, or any suitable combination thereof) that can numerically represent at least some substantive or semantic characteristics of an entirety of the new medical order. Accordingly, the global vector can be considered as an embedding, encoded representation, or latent representation of the new medical order as a whole. In various instances, the vector component can generate the global vector, by applying or executing the vectorizer on the new medical order.

In contrast, a local vector can, despite its name, be any suitable mathematical quantity (e.g., one or more scalars, one or more vectors, one or more matrices, one or more tensors, or any suitable combination thereof) that can numerically represent at least some substantive or semantic characteristics of less than an entirety of the new medical order. Accordingly, a local vector can be considered as an embedding, encoded representation, or latent representation of some portion or part of the new medical order. In various instances, the new medical order can be considered as being made up of a plurality of textual sections (e.g., each discrete text field of the new medical order can be considered as a respective textual section). In various cases, for any given textual section of the new medical order, the vector component can generate a respective local vector, by applying or executing the vectorizer on that given textual section and not on a remainder of the new medical order. In some aspects, for any given combination of two or more, but fewer than all, of the discrete textual sections, the vector component can generate a respective local vector, by applying or executing the vectorizer on that given combination of textual sections and not on a remainder of the new medical order.

Note that, in some cases, the vector component can apply any suitable compression technique (e.g., principal component analysis (PCA)) to the global vector or to the set of local vectors, so as to reduce their sizes (e.g., so as to make such vectors more compact or space-efficient).

Note that the global vector can be considered as capturing coarse semantic characteristics of the new medical order. In contrast, note that each local vector that represents a single respective textual section can be considered as capturing fine or granular semantic characteristics of the new medical order. Furthermore, note that each local vector that represents a respective combination of textual sections (such combinations may also be referred to herein as “prompts”) can be considered as capturing intermediate-granularity semantic characteristics of the new medical order, depending upon the size of the combination (e.g., the local vectors of larger combinations can capture coarser or less fine semantic content; the local vectors of smaller combinations can capture finer or less coarse semantic content). Thus, the global vector and the set of local vectors can collectively be considered as capturing or encompassing the semantic content of the new medical order in a deeper, richer, fuller, or otherwise more enhanced, complete, or nuanced way than any of such vectors could do alone or in isolation. In other words, using both global and local vectors to numerically represent the new medical order can be considered as a technique or strategy that boosts the amount of semantic detail that can be captured by whatever vectorizer is leveraged by the vector component.

In various embodiments, the search component of the computerized tool can electronically store, maintain, control, or otherwise access an historical order-label database. In various aspects, the historical order-label database can comprise a plurality of past medical orders and a respectively corresponding plurality of classification labels that were known or deemed to have been requested or prescribed by those past medical orders. In various instances, the vector component can compute (or can have computed) a respective global vector and a respective set of local vectors for each past medical order in the historical order-label database, using the same vectorization techniques that the vector component applied to the new medical order. Accordingly, in various cases, the search component can search through the historical order-label database, by respectively comparing (e.g., via cosine similarity calculations, via Euclidean distance calculations, via graph computation or clustering techniques, via Facebook AI Similarity Search (FAISS)) the global and local vectors of the new medical order to the global and local vectors of each past medical order. In various aspects, such comparison can yield a respective similarity score for each past medical order, where a similarity score can be a scalar whose magnitude indicates how semantically similar or dissimilar a respective past medical order is to the new medical order. As described herein, the semantic content of medical orders can be more fully, deeply, or richly captured or represented by using both global and local vectors. Thus, similarity scores that are computed using both global and local vectors can be considered as more accurately indicating semantic similarity than would otherwise be possible. In any case, the search component can select a past medical order from the historical order-label database that is sufficiently similar to the new medical order (e.g., the selected past medical order can be whichever past medical order has a highest similarity score with respect to the new medical order). In various aspects, because the selected past medical order and the new medical order can be semantically similar to each other, it can be concluded or expected that whichever classification label in the historical order-label database corresponds to the selected past medical order should also correspond to the new medical order. Thus, the search component can assign a new classification label to the new medical order, where that new classification label can be the same as or identical to whichever classification label corresponds to the selected past medical order, and where that new classification label can be considered as indicating or specifying what specific medical action is requested or prescribed for the medical patient by the new medical order.

In this way, the new medical order can be classified, without having to utilize a dedicated machine learning classifier or a fine-tuned LLM, and thus without having to suffer the concomitant disadvantages of existing techniques (e.g., without having as large a computational footprint as existing techniques; without separate instantiations of the computerized tool having to be implemented for separate medical sites; without having to undergo continual learning or re-training).

In some embodiments, the search component can insert the new medical order, its global and local vectors, and the new classification label as a new entry in the historical order-label database. Accordingly, the global and local vectors of future medical orders can be compared against those of the new medical order as appropriate. In this way, the historical order-label database can be iteratively grown or expanded over time.

In various embodiments, as mentioned above, the medical action that is requested or prescribed by the new medical order and that is specified by the new classification label can be automatically performable by the medical device. Accordingly, in various aspects, the execution component of the computerized tool can, in response to the new classification label, electronically instruct the medical device to automatically perform the requested or prescribed medical action on the medical patient. As a non-limiting example, the medical device can be a medical imaging scanner (e.g., MRI scanner, X-ray scanner), and the requested or prescribed medical action can be a specific imaging protocol (e.g., defining a specific configuration of scanner settings) that can be run by the medical device. So, the execution component can electronically command the medical device to automatically scan the medical patient according to the specific imaging protocol, thereby yielding one or more scanned images of the medical patient. As another non-limiting example, the medical device can be an automated medication dispenser (e.g., a reservoir of fluidic medication that can be intravenously pumped or injected into the medical patient), and the requested or prescribed medical action can be a specific medication dispensation protocol (e.g., defining a specific dosage of fluidic medication) that can be run by the medical device. So, the execution component can electronically command the medical device to automatically dispense medication to the medical patient according to the specific dispensation protocol. As yet another non-limiting example, the medical device can be an automated surgery tool (e.g., a laparoscopic robot), and the requested or prescribed medical action can be a specific surgery protocol (e.g., defining dimensions or anatomical coordinates of a specific incision that is to be made) that can be run by the medical device. So, the execution component can electronically command the medical device to automatically operate on the medical patient according to the specific surgery protocol.

Various embodiments described herein can be employed to use hardware or software to solve problems that are highly technical in nature (e.g., to facilitate global and local search-based classification of text), that are not abstract and that cannot be performed as a set of mental acts by a human. Further, some of the processes performed can be performed by a specialized computer (e.g., text vectorizers, medical devices) for carrying out defined acts related to text classification. For example, such defined acts can include: accessing, by a device operatively coupled to a processor, a new medical order associated with a medical patient; computing, by the device: one or more global vector representations of the new medical order; and one or more local vector representations for respective ones or combinations of a set of textual sections that make up the new medical order, thereby yielding a set of local vector representations of the new medical order; and identifying, by the device, a new classification label for the new medical order, based on searching an historical order-label database using both the set of global vector representations and the set of local vector representations. In some cases, the medical patient can be associated with a medical imaging scanner, the new classification label can specify an imaging protocol for the medical imaging scanner, and the defined acts can comprise: causing, by the device, the medical imaging scanner to scan the medical patient according to the imaging protocol. In other cases, an airway or blood vessel of the medical patient can be coupled to a tank containing a fluidic medication, the new classification label can specify a dosage, and the defined acts can comprise: causing, by the device, a pump of the tank to dispense the fluidic medication to the airway or blood vessel of the medical patient in accordance with the dosage. In yet other cases, the medical patient can be associated with a robotic surgery apparatus, the new classification label can specify a surgical intervention, and the defined acts can comprise: causing, by the device, the robotic surgery apparatus to perform the surgical intervention on the medical patient.

Such defined acts are not performed manually by humans. Indeed, neither the human mind nor a human with pen and paper can: electronically calculate global and local vectors (e.g., embeddings, latent representations) of a textual document (e.g., a medical order); electronically identify a classification label for the textual document by searching an historical database using the global and local vectors together; and electronically command a medical device to automatically perform whatever automated action is specified in the classification label. Indeed, medical devices (e.g., MRI scanners, intravenous medication pumps, laparoscopic robots) are inherently-computerized, hardware-based constructs that simply cannot be meaningfully implemented in any way by the human mind without computers. Additionally, text vectorizers are inherently computerized, software-based constructs that also cannot be meaningfully implemented in any way by the human mind without computers. In fact, text classification is itself a computerized task that is focused on enabling computers to correctly, accurately, or reliably identify or generate classification labels for inputted texts. It would make no sense whatsoever to discuss the computerized task of text classification without regard to computing environments. Accordingly, a computerized tool that can classify text by utilizing both global and local vector representations of that text is likewise inherently-computerized and cannot be implemented in any sensible, practical, or reasonable way without computers.

Moreover, various embodiments described herein can integrate into a practical application various teachings relating to the field of text classification. As described above, when given a medical order, it can be desired to automatically classify that medical order, so as to identify or determine which specific medical action is textually requested or textually prescribed by the given medical order. Existing techniques facilitate such classification via specially trained machine learning classifiers or via fine-tuned LLMs. Unfortunately, such existing techniques are extremely computationally expensive. Indeed, a specially trained classifier or fine-tuned LLM can have a massive computational footprint on its own (e.g., containing millions or billions of learnable internal parameters). Furthermore, existing techniques exacerbate such massive computational footprint by requiring a separate classifier or LLM for each distinct medical site for which medical order classification is desired. Further still, existing techniques require continual learning to handle data drift, but such continual learning is incredibly time-consuming (e.g., in terms of training data collation, number of training epochs, and regulatory re-compliance or re-verification) and vulnerable to catastrophic forgetting. Thus, existing techniques are disadvantageous.

Various embodiments described herein can address one or more of these technical problems. In particular, the present inventors devised various techniques for facilitating text classification that leverage both global and local vector representations of text. In particular, for any given medical order, a global vector representing the entirety of the given medical order can be computed (e.g., via TF-IDF, or via an encoder of any pre-trained text-analysis model), local vectors representing individual ones or combinations of discrete textual sections of the given medical order can be computed, and those global and local vectors can be used to search (e.g., via cosine similarity computations) through an order-label database for a past medical order that is semantically similar to the given medical order. When that past medical order is found, it can be expected that whatever classification label that was previously assigned to that past medical order should likewise be assigned to the given medical order.

Note that various embodiments described herein can avoid, reduce, or otherwise ameliorate the technical problems of existing techniques.

First, various embodiments can have a smaller (e.g., in some cases, several orders of magnitude smaller) computational footprint than existing techniques. Indeed, the order-label database of various embodiments described herein can provide good classification performance with as few as a couple hundred past medical orders. Such an order-label database can consume thousands of times less computer memory or storage space than dedicated classifiers or LLMs which can take up thousands of gigabytes of memory. Moreover, embodiments described herein that compute global and local vectors via non-machine-learning vectorizers (e.g., TF-IDF) can have no trainable internal parameters at all, unlike the millions or billions of trainable internal parameters of existing techniques. Although embodiments described herein that compute global and local vectors via machine learning vectorizers (e.g., pre-trained encoders from LLMs or other text-analysis models) do include non-zero numbers of trainable internal parameters, such cardinality of trainable internal parameters pales in comparison to that of existing techniques. After all, any type of machine learning model that is configured or trained to analyze text (e.g., an LLM, a text classifier, a text segmenter, a text regressor, a variational autoencoder) can be considered as being made up of an encoder and a head. The encoder can be considered as whatever upstream portion or layers of that model that convert inputted text to numerical representations (e.g., to embeddings or latent vectors), and the head can be whatever downstream portion or layers of that model that perform some inferencing task (e.g., classification, segmentation, regression, synthesis) based on those numerical representations. Accordingly, the encoder of the model necessarily has a smaller (e.g., in some cases, a mere fraction of the) computational footprint of the model itself. Accordingly, embodiments described herein that utilize machine learning vectorizers nevertheless exhibit significantly smaller computational footprints than existing techniques.

Next, various embodiments described herein do not require a distinct order-label database and a distinct vectorizer for each distinct medical site (e.g., for each different hospital) for which medical order classification is desired to be provided. Indeed, as mentioned above, the semantic characteristics of medical orders can vary widely across different medical sites (e.g., different hospitals serve different populations of patients using different prescribed treatments and different wordings or phraseologies). It has been found that a single, centralized classifier or LLM is not, even after significant training, able to reliably or confidently learn or handle such wide semantic variety of medical orders (e.g., such single, centralized classifier or LLM often learns how to provide only mediocre medical order classification accuracy across all of such different medical sites). To address this, existing techniques deploy a separate instantiation of the classifier or LLM that is tailored to the specific medical order characteristics of each respective medical site. This multiplies the already-massive computational footprint of existing techniques. In stark contrast, various embodiments described herein do not require a separate database-and-vectorizer instantiation for each respective medical site (e.g., in some cases, a centralized order-label database and vectorizer can be used across medical sites; in other cases, distinct order-label databases can be used for each medical site, but a centralized vectorizer can nevertheless be used across all medical sites). This can be due to the implementation of both global and local vector representations of medical orders. After all, as mentioned above, the present inventors recognized that a global vector of a medical order is oftentimes not equivalent or identical to an aggregation of local vectors of that medical order, even when that global vector and those local vectors are created by the same vectorizer. In other words, the present inventors realized that global vectors and local vectors capture different types or kinds of semantic information and can thus be considered as complementary to each other. Stated differently, a global vector can capture or encapsulate semantic information that is hidden from or that cannot be contained in a local vector; likewise, a local vector can capture or encapsulate semantic information that is hidden from or that cannot be contained in a global vector. So, the present inventors realized that representing any given medical order with both global and local vectors can be considered as a way to more fully or completely capture the semantic content of that given medical order. Thus, global and local vectors can, when used together, be able to better handle the wide variety of medical order semantic characteristics that arises across different medical sites, unlike existing techniques.

Additionally, various embodiments described herein do not involve re-training, unlike existing techniques. After all, embodiments that compute global and local vectors via non-machine-learning vectorizers can have no learnable internal parameters to which re-training could possibly be applied. Moreover, although embodiments that compute global and local vectors via machine learning vectorizers (e.g., pre-trained encoders) do have learnable internal parameters to which re-training could possibly be applied, such re-training can be completely eschewed. Indeed, as mentioned above, the purpose of re-training can be to learn how to handle medical orders whose semantic characteristics have drifted from those involved in original training. But, various embodiments described herein can handle such drift by mere insertion or addition of a small number of drifted exemplars into the herein-described order-label database. For example, when any drifted medical order and its accompanying classification label is added to the order-label database, its global and local vectors can be computed. So, that drifted medical order can thus be leveraged by the herein-described search component to classify any future medical orders that are semantically similar to it (e.g., that are drifted in the same way). Since re-training can be eschewed, various embodiments described herein do not require the collection and annotation of many thousands of drifted medical orders, do not require tens of hours spent during backpropagation, can be not subject to burdensome regulatory re-compliance checks, and can avoid the problem of catastrophic forgetting.

Accordingly, various embodiments described herein can be considered as a clever or inventive utilization of text vectors that provides computationally light-weight text classification and that is not afflicted by the problems of continual learning. Thus, various embodiments described herein certainly constitute a tangible and concrete technical improvement or technical advantage in the field of text classification. Accordingly, such embodiments clearly qualify as useful and practical applications of computers.

Furthermore, various embodiments described herein can control real-world tangible devices based on the disclosed teachings. For example, various embodiments described herein can identify a classification label for any given medical order, where that classification label specifies or indicates a particular medical action (e.g., particular scanning protocol, particular medication dosage, particular surgical procedure) that is automatically performable by a real-world medical device (e.g., CT scanner, medication dispenser, laparoscopic robot). Accordingly, various embodiments described herein can electronically cause the real-world medical device to automatically perform, initiate, or otherwise carry out such particular medical action.

It should be appreciated that the herein figures and description provide non-limiting examples of various embodiments and are not necessarily drawn to scale.

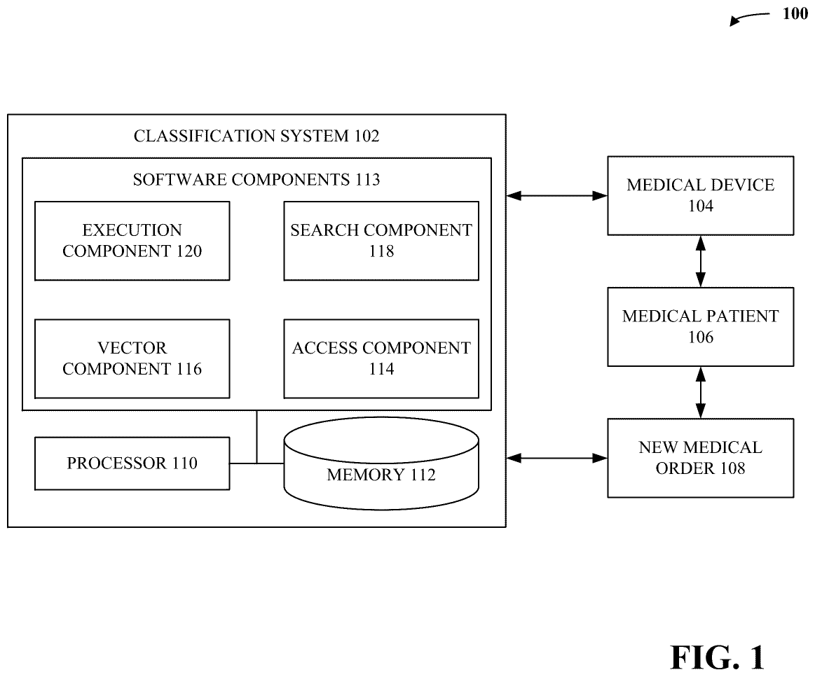

FIG. 1 illustrates a block diagram of an example, non-limiting system 100 that can facilitate global and local search-based classification of text in accordance with one or more embodiments described herein. As shown, a classification system 102 can be electronically integrated, via any suitable wired or wireless electronic connection, with a medical device 104 or with a new medical order 108.

In various embodiments, the medical device 104 can be any suitable type of computerized medical equipment or computerized medical modality that can electronically monitor any suitable biological, clinical, or medical attribute, characteristic, or feature of a medical patient 106 or that can otherwise electronically perform any suitable automated medical action for, on, or with respect to the medical patient 106.

In various aspects, the medical device 104 can be any suitable computerized equipment or modality for capturing or generating medical images of the medical patient 106. As a non-limiting example, the medical device 104 can be a CT scanner that can capture or generate CT scanned pixel arrays or voxel arrays depicting any suitable anatomical structure (e.g., tissue, organ, body part, body cavity, or portion thereof) of the medical patient 106. As another non-limiting example, the medical device 104 can be an MRI scanner that can capture or generate MRI scanned pixel arrays or voxel arrays depicting any suitable anatomical structure of the medical patient 106. As even another non-limiting example, the medical device 104 can be an X-ray scanner that can capture or generate X-ray scanned pixel arrays or voxel arrays depicting any suitable anatomical structure of the medical patient 106. As yet another non-limiting example, the medical device 104 can be an ultrasound scanner that can capture or generate ultrasound scanned pixel arrays or voxel arrays depicting any suitable anatomical structure of the medical patient 106. As still another non-limiting example, the medical device 104 can be a PET scanner that can capture or generate PET scanned pixel arrays or voxel arrays depicting any suitable anatomical structure of the medical patient 106. As another non-limiting example, the medical device 104 can be an NM scanner that can capture or generate NM scanned pixel arrays or voxel arrays depicting any suitable anatomical structure of the medical patient 106.

In various instances, the medical device 104 can be any suitable computerized equipment or modality for automatically dispensing, administering, or injecting medication to the medical patient 106. As a non-limiting example, the medical device 104 can comprise a tank or reservoir that is filled with an aerosolized or gaseous medication. In such case, the tank or reservoir can be coupled by medical tubing to an airway of the medical patient 106 (e.g., such medical tubing can be inserted into a nose or mouth of the medical patient 106; such medical tubing can be routed to a face mask of the medical patient 106). In various aspects, an actuatable motor or pump can be operatively coupled to the tank or reservoir, such that activation of the motor or pump can cause a selectively controlled amount of the aerosolized or gaseous medication to be delivered from the tank or reservoir, through the medical tubing, and into the airway of the medical patient 106. As another non-limiting example, the medical device 104 can comprise a tank or reservoir that is filled with a liquid medication. In such case, the tank or reservoir can be coupled by medical tubing to a blood vessel of the medical patient 106 (e.g., such medical tubing can be inserted via a needle into a vein or artery of the medical patient 106). In various instances, an actuatable motor or pump can be operatively coupled to the tank or reservoir, such that activation of the motor or pump can cause a selectively controlled amount of the liquid medication to be delivered from the tank or reservoir, through the medical tubing, and into the blood vessel of the medical patient 106. As yet another non-limiting example, the medical device 104 can comprise a tank or reservoir that is filled with medication tablets (e.g., pills or lozenges). In such case, the tank or reservoir can have a dispensation chute or slide that leads to a food tray or pill tray of the medical patient 106. In various aspects, an actuatable motor can be operatively coupled to the tank or reservoir, such that activation of the motor can cause a selectively controlled number of the medication tablets to be delivered from the tank or reservoir, down the dispensation chute or slide, and into the food tray or pill tray of the medical patient 106.

In various cases, the medical device 104 can be any suitable computerized equipment or modality for automatically performing surgical operations or interventions on the medical patient 106. Indeed, in various aspects, the medical device 104 can comprise any suitable number of any suitable types of actuatable robotic arms or end effectors that can automatically: make surgical incisions into or on any suitable anatomical structure or portion thereof of the medical patient 106; excise any suitable anatomical structure or portion thereof from the medical patient 106; ablate any suitable anatomical structure or portion thereof the medical patient 106; apply stitching to any suitable anatomical structure or portion thereof the medical patient 106; or insert implants or prostheses into any suitable anatomical structure or portion thereof of the medical patient 106. As some non-limiting examples, the medical device 104 can configured or designed to automatically perform any of the following on or for the medical patient 106: automated ophthalmologic surgical procedures (e.g., pterygium repairs, vitreoretinal operations); automated cardiovascular surgical procedures (e.g., atrial septal defect repair, mitral valve repair, coronary artery bypass); automated thoracic surgical procedures (e.g., mediastinal mass resection, lung mass resection); automated ear surgical procedures (e.g., cochlear implantation); automated abdominal surgical procedures (e.g., laparoscopic surgery, bariatric surgery, gastrectomy, pancreatectomy, hernia repair); automated orthopedic surgical procedures (e.g., bone arthroplasty); or automated spinal surgical procedures (e.g., spinal screw insertion).

In various aspects, the medical device 104 can be or comprise any suitable combination of any of the aforementioned.

In various embodiments, the new medical order 108 can be any suitable electronic document (e.g., written in sentences or sentence fragments) that textually orders, requests, calls for, or otherwise prescribes the performance of any suitable medical action on or with respect to the medical patient 106. In various aspects, the new medical order 108 can further textually indicate, describe, or explain any suitable demographic information regarding the medical patient 106. As some non-limiting examples, such demographic information can include: body mass index of the medical patient 106; age of the medical patient 106; gender or sex of the medical patient 106; height of the medical patient 106; occupation of the medical patient 106; or ethnicity of the medical patient 106. In various instances, the new medical order 108 can further textually indicate, describe, or explain any suitable lifestyle information of the medical patient 106. As some non-limiting examples, such lifestyle information can include: smoking habits of the medical patient 106; alcohol consumption habits of the medical patient 106; exercise habits of the medical patient 106; or dietary habits of the medical patient 106. In various cases, the new medical order 108 can further textually indicate, describe, or explain any suitable medical information regarding the medical patient 106. As some non-limiting examples, such medical information can include: allergies of the medical patient 106; surgical history of the medical patient 106; medication history of the medical patient 106; symptoms reported by the medical patient 106; medical test results regarding the medical patient 106; or medical professional observations regarding the medical patient 106. In various instances, the new medical order 108 can further textually indicate, describe, or explain any other suitable metadata as desired, such as the identity of the hospital department (e.g., radiology department, gastroenterology department, physical therapy department) from which the new medical order 108 has originated.

In various aspects, the new medical order 108 can be typed or written (e.g., using any suitable word-processing software on any suitable computing device) by a medical professional (e.g., physician, nurse, medical device technician) that is attending to or otherwise responsible for overseeing the medical patient 106. Accordingly, in various cases, the new medical order 108 can be considered as an electronic textual document, paper, or form that indicates various information about the medical patient 106 and that specifies whatever particular medical action that an attending physician of the medical patient 106 has requested in order to diagnose or treat the medical patient 106. In some cases, the particular medical action can be any suitable protocol or procedure that is automatically performable by the medical device 104. As a non-limiting example, suppose that the medical device 104 is a medical imaging scanner. In such case, the particular medical action can be a specific scanning protocol that can be run by the medical device 104. As another non-limiting example, suppose that the medical device 104 is a medication dispenser. In such case, the particular medical action can be dispensation of a particular dosage of medication that can be implemented by the medical device 104. As yet another non-limiting example, suppose that the medical device 104 is a robotic surgery apparatus. In such case, the particular medical action can be a specific surgical procedure that can be run by the medical device 104.

In any case, it can be desired to automatically classify the new medical order 108, so as to determine or identify what particular medical action it requests, calls for, or prescribes with respect to the medical patient 106. As described herein, the classification system 102 can accomplish such classification.

In various embodiments, the classification system 102 can comprise a processor 110 (e.g., computer processing unit, microprocessor) and a non-transitory computer-readable memory 112 that is operably or operatively or communicatively connected or coupled to the processor 110. The non-transitory computer-readable memory 112 can store computer-executable instructions which, upon execution by the processor 110, can cause the processor 110 or other components of the classification system 102 (e.g., access component 114, vector component 116, search component 118, execution component 120) to perform one or more acts. In various embodiments, the non-transitory computer-readable memory 112 can store computer-executable components (e.g., access component 114, vector component 116, search component 118, execution component 120), and the processor 110 can execute the computer-executable components.

In various embodiments, the classification system 102 can comprise an access component 114. In various aspects, the access component 114 can electronically access or otherwise electronically communicate in any suitable fashion with the medical device 104. Accordingly, the access component 114 can electronically transmit any suitable electronic data to the medical device 104, and the medical device 104 can likewise electronically transmit any suitable electronic data to the access component 114. In some instances, the access component 114 can be considered as a proxy or conduit through which other components of the classification system 102 can interact with, communicate with, or otherwise manipulate the medical device 104. In various aspects, the access component 114 can electronically access the new medical order 108. That is, the access component 114 can electronically receive, electronically retrieve, or otherwise electronically obtain the new medical order 108, from any suitable electronic source or database (e.g., possibly from the medical device 104 or from an associated computerized workstation). In any case, the access component 114 can be considered as a proxy or conduit through which other components of the classification system 102 can interact with, control, or otherwise manipulate the new medical order 108. However, these are mere non-limiting examples. In other cases, the access component 114 can be omitted, and any other components of the classification system 102 can communicate or interact directly with the medical device 104 or with the new medical order 108.

In various embodiments, the classification system 102 can comprise a vector component 116. In various aspects, the vector component 116 can, as described herein, leverage one or more vectorizers so as to generate global vectors and local vectors to semantically represent the new medical order 108.

In various embodiments, the classification system 102 can comprise a search component 118. In various instances, the search component 118 can, as described herein, assign a new classification label to the new medical order 108, by searching an historical order-label database according to the global and local vectors representing the new medical order 108. In various cases, the new classification label can identify whatever medical action is requested, called for, or prescribed by the new medical order 108.

In various embodiments, the classification system 102 can comprise an execution component 120. In various cases, the execution component 120 can, as described herein, instruct the medical device 104 to automatically perform whatever medical action is indicated by the new classification label.

Note that, in various instances, the access component 114, the vector component 116, the search component 118, and the execution component 120 can collectively be considered as being one or more software components 113 of the classification system 102. In various aspects, it should be appreciated that the one or more software components 113 are described primarily herein as comprising four components (e.g., the access component 114, the vector component 116, the search component 118, and the execution component 120) for ease of explanation and illustration. However, the one or more software components 113 are not limited to being implemented as exactly such four components in every embodiment. Indeed, in some embodiments, the functionalities described herein of such four components can be combined in any suitable fashions, so as to be implemented in or by fewer than four components (e.g., in some cases, a single component can perform all of the functionalities that are described herein with respect to the access component 114, the vector component 116, the search component 118, and the execution component 120). In other embodiments, the functionalities described herein of such four components can instead be distributed, separated, split, or fragmented in any suitable fashions, so as to be implemented in or by more than four components (e.g., two or more components can facilitate the functionalities that are performable by the access component 114; two or more components can facilitate the functionalities that are performable by the vector component 116; two or more components can facilitate the functionalities that are performable by the search component 118; two or more components can facilitate the functionalities that are performable by the execution component 120).

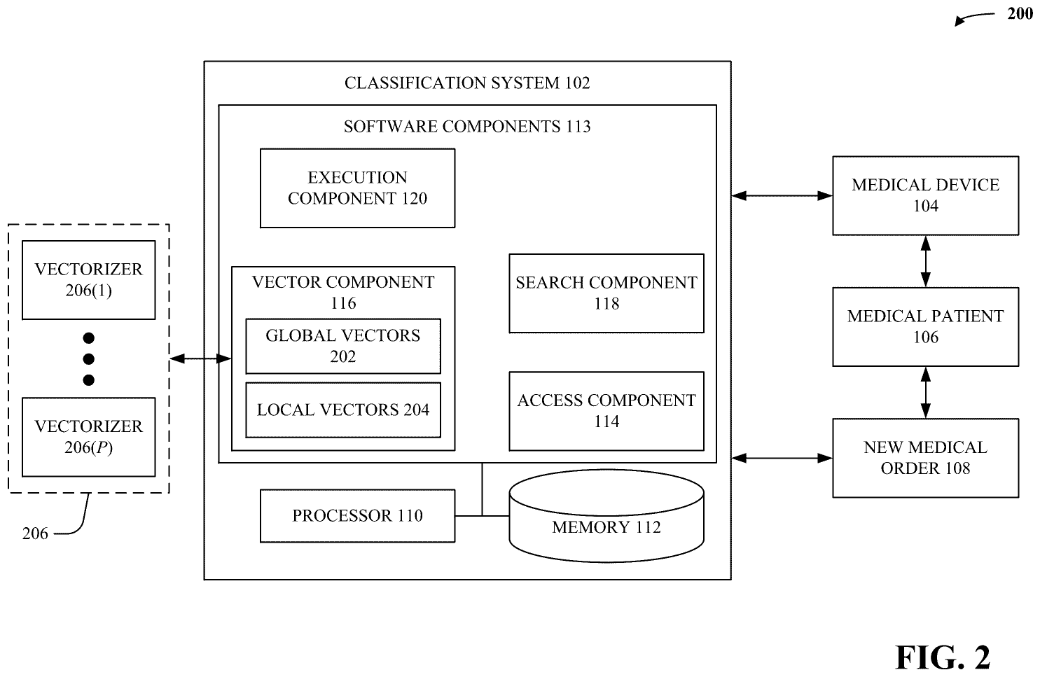

FIG. 2 illustrates a block diagram of an example, non-limiting system 200 including global vectors and local vectors that can facilitate global and local search-based classification of text in accordance with one or more embodiments described herein. As shown, the system 200 can, in some cases, comprise the same components as the system 100, and can further comprise one or more global vectors 202, a set of local vectors 204, and one or more vectorizers 206.

In various embodiments, the vector component 116 can electronically store, electronically maintain, electronically control, or otherwise electronically access the one or more vectorizers 206. In various aspects, the one or more vectorizers 206 can comprise p vectorizers, for any suitable positive integer p: a vectorizer 206(1) to a vectorizer 206(p). In various instances, each of the one or more vectorizers 206 can be a unique, distinct, or separate vectorization technique or vectorization algorithm that can convert text into numerical representations (e.g., into scalars, vectors, matrices, tensors, or any suitable combination thereof). So, the vectorizer 206(1) can be a first vectorization technique or algorithm that can transform inputted text into numbers and that can be unique among the one or more vectorizers 206 (e.g., that can be different from any others of the one or more vectorizers 206). Likewise, the vectorizer 206(p) can be a p-th vectorization technique or algorithm that can transform inputted text into numbers and that can be unique among the one or more vectorizers 206. In various aspects, any of the one or more vectorizers 206 can be any suitable non-machine-learning vectorization techniques or algorithms (e.g., techniques or algorithms that do not include trainable parameters). Some non-limiting examples of such non-machine-learning vectorization techniques or algorithms can include: a TF-IDF vectorization technique or algorithm; a one-hot encoding vectorization technique or algorithm; a count vectorization technique or algorithm; a bag-of-words vectorization technique or algorithm; or an n-grams vectorization technique or algorithm. In various instances, any of the one or more vectorizers 206 can be any suitable machine learning vectorization techniques or algorithms. Some non-limiting examples of such machine learning vectorization techniques or algorithms can include: an encoder from a pre-trained text variational autoencoder (e.g., the variational autoencoder can comprise an encoder and a decoder, where the trainable internal parameters (convolutional kernels, weight matrices, attention modules, long-short-term memory weights) of the encoder convert inputted text to numerical representations, and where the trainable internal parameters of the decoder reconstruct the inputted text based on those numerical representations); an encoder from a pre-trained text classifier (e.g., the text classifier can comprise an encoder and a head, where the trainable internal parameters of the encoder convert inputted text to numerical representations, and where the trainable internal parameters of the head classify the inputted text based on those numerical representations); an encoder from a pre-trained text segmenter (e.g., the text segmenter can comprise an encoder and a head, where the trainable internal parameters of the encoder convert inputted text to numerical representations, and where the trainable internal parameters of the head segment the inputted text based on those numerical representations); an encoder from a pre-trained text regressor (e.g., the text regressor can comprise an encoder and a head, where the trainable internal parameters of the encoder convert inputted text to numerical representations, and where the trainable internal parameters of the head compute some regression output based on those numerical representations); or an encoder from a pre-trained LLM (e.g., the LLM can comprise an encoder and a synthesizer, where the trainable internal parameters of the encoder convert inputted text to numerical representations, and where the trainable internal parameters of the synthesizer create synthetic textual content based on those numerical representations). Note that, in various cases, any of the one or more vectorizers 206 can be an encoder from any machine learning model that has been pre-trained to analyze text, regardless of whether that machine learning model is or was configured to analyze medical orders (e.g., the encoder from any pre-trained text-classification model can suffice, even models that have never encountered medical orders).

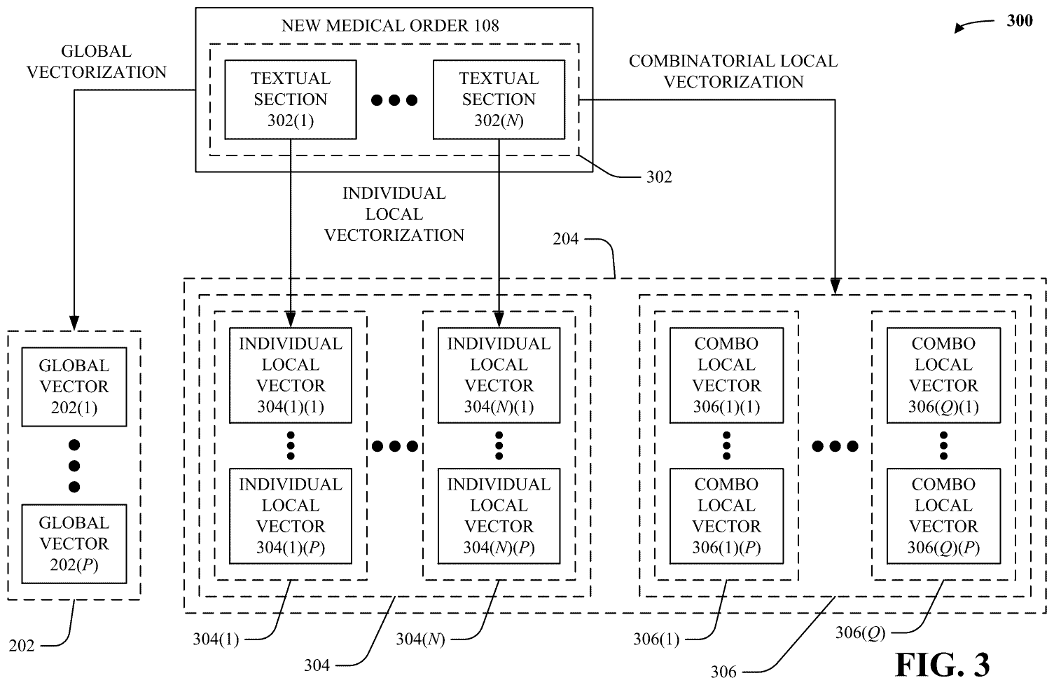

In various cases, the vector component 116 can electronically generate the one or more global vectors 202 and the set of local vectors 204, by applying or executing the one or more vectorizers 206 to or on the new medical order 108. Non-limiting aspects are described with respect to FIG. 3.

FIG. 3 illustrates an example, non-limiting block diagram 300 showing how the one or more global vectors 202 and the set of local vectors 204 can be generated in accordance with one or more embodiments described herein.

In various embodiments, as shown, the new medical order 108 can be considered as comprising a plurality of textual sections 302. In various aspects, the plurality of textual sections 302 can comprise n sections, for any suitable positive integer n>1: a textual section 302(1) to a textual section 302(n). In various instances, each of the plurality of textual sections 302 can be any suitable discrete part or portion of the new medical order 108. As a non-limiting example, each of the plurality of textual sections 302 can be a respective or distinct text field of the new medical order 108 into which whatever medical professional that is attending to the medical patient 106 typed or wrote various text regarding the medical patient 106 or regarding the medical device 104. For instance, the textual section 302(1) can be a first text field of the new medical order 108 into which the attending medical professional typed or wrote one or more first sentences or sentence fragments (e.g., can be an “allergies” text field of the new medical order 108). Likewise, the textual section 302(n) can be an n-th text field of the new medical order 108 into which the attending medical professional typed or wrote one or more n-th sentences or sentence fragments (e.g., can be an “observations” text field of the new medical order 108). Note that, in various cases, the plurality of textual sections 302 can be disjoint or non-overlapping with each other (e.g., each textual section can be a unique, distinct, or separate text field of the new medical order 108).

Now, in various embodiments, the vector component 116 can electronically generate the one or more global vectors 202, by applying or executing the one or more vectorizers 206 to or on the new medical order 108 in its entirety.

As a non-limiting example, the vector component 116 can electronically apply or execute the vectorizer 206(1) on the entirety of the new medical order 108. In various cases, such application or execution can cause the vectorizer 206(1) to compute, produce, or otherwise yield a global vector 202(1). In various aspects, the global vector 202(1) can, despite its name, be one or more scalars, one or more vectors, one or more matrices, one or more tensors, or any suitable combination thereof that numerically represent any suitable semantic characteristics or properties of the entirety of the new medical order 108. Note that the term “global” can be considered as appropriate, since the global vector 202(1) represents the global entirety of the new medical order 108.

As another non-limiting example, the vector component 116 can electronically apply or execute the vectorizer 206(p) on the entirety of the new medical order 108. In various cases, such application or execution can cause the vectorizer 206(p) to compute, produce, or otherwise yield a global vector 202(p). As above, the global vector 202(p) can, despite its name, be one or more scalars, one or more vectors, one or more matrices, one or more tensors, or any suitable combination thereof that numerically represent any suitable semantic characteristics or properties of the entirety of the new medical order 108.

In various aspects, the global vector 202(1) to the global vector 202(p) can collectively be considered as the one or more global vectors 202. In various instances, each of the one or more global vectors 202 can have the same dimensionality (e.g., the same cardinality of numerical elements) as each other.

Note that, because the one or more vectorizers 206 can be different from each other, each of the one or more vectorizers 206 can be considered as having a unique vectorization perspective. In other words, each of the one or more vectorizers 206 can be considered capturing a unique type or kind of semantic content (e.g., the type or kind of semantic content the vectorizer 206(1) captures can be non-identical to that which the vectorizer 206(p) captures). Accordingly, although each of the one or more global vectors 202 can have the same dimensionality as each other, they can be considered as representing distinct or unique semantic characteristics or properties of the entirety of the new medical order 108 (e.g., they can be considered as distinct semantic snap-shots of the entirety of the new medical order 108).

Moving on, in various embodiments, the vector component 116 can electronically generate the set of local vectors 204, by applying or executing the one or more vectorizers 206 to or on the new medical order 108 not in its entirety.

Indeed, in some cases, the vector component 116 can electronically apply or execute the one or more vectorizers 206 individually on each of the plurality of textual sections 302. In various instances, such application or execution can yield a set of individual local vectors 304.

As a non-limiting example, the vector component 116 can electronically apply or execute the vectorizer 206(1) on the textual section 302(1) alone (e.g., omitting the remaining textual sections of the new medical order 108). In various cases, such application or execution can cause the vectorizer 206(1) to compute, produce, or otherwise yield an individual local vector 304(1)(1). In various aspects, the individual local vector 304(1)(1) can, despite its name, be one or more scalars, one or more vectors, one or more matrices, one or more tensors, or any suitable combination thereof that numerically represent any suitable semantic characteristics or properties of the textual section 302(1) (e.g., and not of the remainder of the new medical order 108). Note that the term “local” can be considered as appropriate, since the individual local vector 304(1)(1) represents only a local part or portion (e.g., 302(1)) of the new medical order 108. Furthermore, note that the term “individual” can be considered as appropriate, since the individual local vector 304(1)(1) represents only an individual one of the plurality of textual sections 302.

As another non-limiting example, the vector component 116 can electronically apply or execute the vectorizer 206(p) on the textual section 302(1) alone. In various cases, such application or execution can cause the vectorizer 206(p) to compute, produce, or otherwise yield an individual local vector 304(1)(p). In various aspects, the individual local vector 304(1)(p) can, despite its name, be one or more scalars, one or more vectors, one or more matrices, one or more tensors, or any suitable combination thereof that numerically represent any suitable semantic characteristics or properties of the textual section 302(1).

In various aspects, the individual local vector 304(1)(1) to the individual local vector 304(1)(p) can collectively be considered as one or more individual local vectors 304(1). In various instances, each of the one or more individual local vectors 304(1) can have the same dimensionality as each other (and thus as the one or more global vectors 202).

Note that, as above, because the one or more vectorizers 206 can be considered as having a unique vectorization perspective, each of the one or more individual local vectors 304(1) can be considered as representing distinct or unique semantic characteristics or properties of the textual section 302(1) (e.g., each can be considered as a unique semantic snap-shot of the textual section 302(1)).

As yet another non-limiting example, the vector component 116 can electronically apply or execute the vectorizer 206(1) on the textual section 302(n) alone. In various cases, such application or execution can cause the vectorizer 206(1) to compute, produce, or otherwise yield an individual local vector 304(n)(1). In various aspects, the individual local vector 304(n)(1) can, despite its name, be one or more scalars, one or more vectors, one or more matrices, one or more tensors, or any suitable combination thereof that numerically represent any suitable semantic characteristics or properties of the textual section 302(n).

As still another non-limiting example, the vector component 116 can electronically apply or execute the vectorizer 206(p) on the textual section 302(n) alone. In various cases, such application or execution can cause the vectorizer 206(p) to compute, produce, or otherwise yield an individual local vector 304(n)(p). In various aspects, the individual local vector 304(n)(p) can, despite its name, be one or more scalars, one or more vectors, one or more matrices, one or more tensors, or any suitable combination thereof that numerically represent any suitable semantic characteristics or properties of the textual section 302(n).

In various aspects, the individual local vector 304(n)(1) to the individual local vector 304(n)(p) can collectively be considered as one or more individual local vectors 304(n). In various instances, each of the one or more individual local vectors 304(n) can have the same dimensionality as each other (and thus as the one or more global vectors 202 and as the one or more individual local vectors 304(1)).

Note that, as above, because the one or more vectorizers 206 can be considered as having a unique vectorization perspective, each of the one or more individual local vectors 304(n) can be considered as representing distinct or unique semantic characteristics or properties of the textual section 302(n) (e.g., each can be considered as a unique semantic snap-shot of the textual section 302(n)).

In various cases, the one or more individual local vectors 304(1) to the one or more individual local vectors 304(n) can collectively be considered as the set of individual local vectors 304.

Now, in some embodiments, there can be a total of

$${\sum }_{i = 2}^{n - 1} ( \begin{matrix}n \\ i\end{matrix} )$$

textual section combinations that include at least two but fewer than all of the plurality of textual sections 302. In various aspects, the vector component 116 can select or choose any q of those possible textual section combinations, for any suitable positive integer

$$q \leq {\sum }_{i = 2}^{n - 1} ( \begin{matrix}n \\ i\end{matrix} ) :$$

a first textual section combination (also referred to as a first prompt) including at least two but fewer than all of the plurality of textual sections 302, to a q-th textual section combination (also referred to as a q-th prompt) including at least two but fewer than all of the plurality of textual sections 302. In various cases, the vector component 116 can electronically apply or execute the one or more vectorizers 206 on each of such q textual section combinations (each of such q prompts). In various instances, such application or execution can yield a set of combo local vectors 306.

As a non-limiting example, the vector component 116 can electronically apply or execute the vectorizer 206(1) on the first textual section combination alone (e.g., omitting whatever portions of the new medical order 108 are not in that first textual section combination). In various cases, such application or execution can cause the vectorizer 206(1) to compute, produce, or otherwise yield a combo local vector 306(1)(1). In various aspects, the combo local vector 306(1)(1) can, despite its name, be one or more scalars, one or more vectors, one or more matrices, one or more tensors, or any suitable combination thereof that numerically represent any suitable semantic characteristics or properties of that first textual section combination (e.g., and not of whatever portions of the new medical order 108 are excluded from that first textual section combination). Note that the term “combo” can be considered as appropriate, since the combo local vector 306(1)(1) represents a distinct combination of the plurality of textual sections 302 rather than an individual one of the plurality of textual sections 302.

As another non-limiting example, the vector component 116 can electronically apply or execute the vectorizer 206(p) on that first textual section combination alone. In various cases, such application or execution can cause the vectorizer 206(p) to compute, produce, or otherwise yield a combo local vector 306(1)(p). In various aspects, the combo local vector 306(1)(p) can, despite its name, be one or more scalars, one or more vectors, one or more matrices, one or more tensors, or any suitable combination thereof that numerically represent any suitable semantic characteristics or properties of that first textual section combination.

In various aspects, the combo local vector 306(1)(1) to the combo local vector 306(1)(p) can collectively be considered as one or more combo local vectors 306(1). In various instances, each of the one or more combo local vectors 306(1) can have the same dimensionality as each other (and thus as the one or more global vectors 202, and as the set of individual local vectors 304).

Note that, as above, because the one or more vectorizers 206 can be considered as having a unique vectorization perspective, each of the one or more combo local vectors 306(1) can be considered as representing distinct or unique semantic characteristics or properties of that first textual section combination (e.g., each can be considered as a unique semantic snap-shot of that first textual section combination).

As yet another non-limiting example, the vector component 116 can electronically apply or execute the vectorizer 206(1) on the q-th textual section combination alone (e.g., omitting whatever portions of the new medical order 108 are not in that q-th textual section combination). In various cases, such application or execution can cause the vectorizer 206(1) to compute, produce, or otherwise yield a combo local vector 306(q)(1). In various aspects, the combo local vector 306(q)(1) can, despite its name, be one or more scalars, one or more vectors, one or more matrices, one or more tensors, or any suitable combination thereof that numerically represent any suitable semantic characteristics or properties of that q-th textual section combination (e.g., and not of whatever portions of the new medical order 108 are excluded from that q-th textual section combination).

As even another non-limiting example, the vector component 116 can electronically apply or execute the vectorizer 206(p) on that q-th textual section combination alone. In various cases, such application or execution can cause the vectorizer 206(p) to compute, produce, or otherwise yield a combo local vector 306(q)(p). In various aspects, the combo local vector 306(q)(p) can, despite its name, be one or more scalars, one or more vectors, one or more matrices, one or more tensors, or any suitable combination thereof that numerically represent any suitable semantic characteristics or properties of that q-th textual section combination.

In various aspects, the combo local vector 306(q)(1) to the combo local vector 306(q)(p) can collectively be considered as one or more combo local vectors 306(q). In various instances, each of the one or more combo local vectors 306(q) can have the same dimensionality as each other (and thus as the one or more global vectors 202, and as the set of individual local vectors 304, and as the one or more combo local vectors 306(1)).

Note that, as above, because the one or more vectorizers 206 can be considered as having a unique vectorization perspective, each of the one or more combo local vectors 306(q) can be considered as representing distinct or unique semantic characteristics or properties of that q-th textual section combination (e.g., each can be considered as a unique semantic snap-shot of that q-th textual section combination).

In various cases, the one or more combo local vectors 306(1) to the one or more combo local vectors 306(q) can collectively be considered as the set of combo local vectors 306. Furthermore, in various aspects, the set of individual local vectors 304 and the set of combo local vectors 306 can collectively be considered as forming the set of local vectors 204.

Note that the one or more global vectors 202 and the set of local vectors 204 can together be considered as collectively capturing, encapsulating, or otherwise representing various semantic characteristics or properties of the new medical order 108 from various different scopes, scales, or levels of granularity. Indeed, some of such vectors (e.g., 202) can be considered as representing coarse semantic characteristics of the new medical order 108, whereas others of such vectors (e.g., 304) can be considered as representing fine semantic characteristics of the new medical order 108, and whereas yet others of such vectors (e.g., 306) can be considered as representing intermediate-scale semantic characteristics of the new medical order 108. Thus, the one or more global vectors 202 and the set of local vectors 204 can together be considered as richly or deeply capturing the semantic content of the new medical order 108.

Although FIG. 3 shows that each of the one or more vectorizers 206 can be executed or applied to each granularity scale of the new medical order 108 (e.g., such that all of 202, 304(1), 304(n), 306(1), and 306(q) have a cardinality of p), this is a mere non-limiting example for ease of explanation and illustration. It is to be appreciated that different ones of the one or more vectorizers 206 can be applied or executed at different granularity scales to the new medical order 108 (e.g., different subsets of 206 can be used to generate different ones of 202, 304(1), 304(n), 306(1), and 306(q); such that 202, 304(1), 304(n), 306(1), and 306(q) have different cardinalities all of which are less than or equal to p).

In some cases, the vector component 116 can electronically compress or reduce the dimensionalities of the one or more global vectors 202 and of the set of local vectors 204 via any suitable techniques. A non-limiting example of such compression or reduction technique can be PCA.

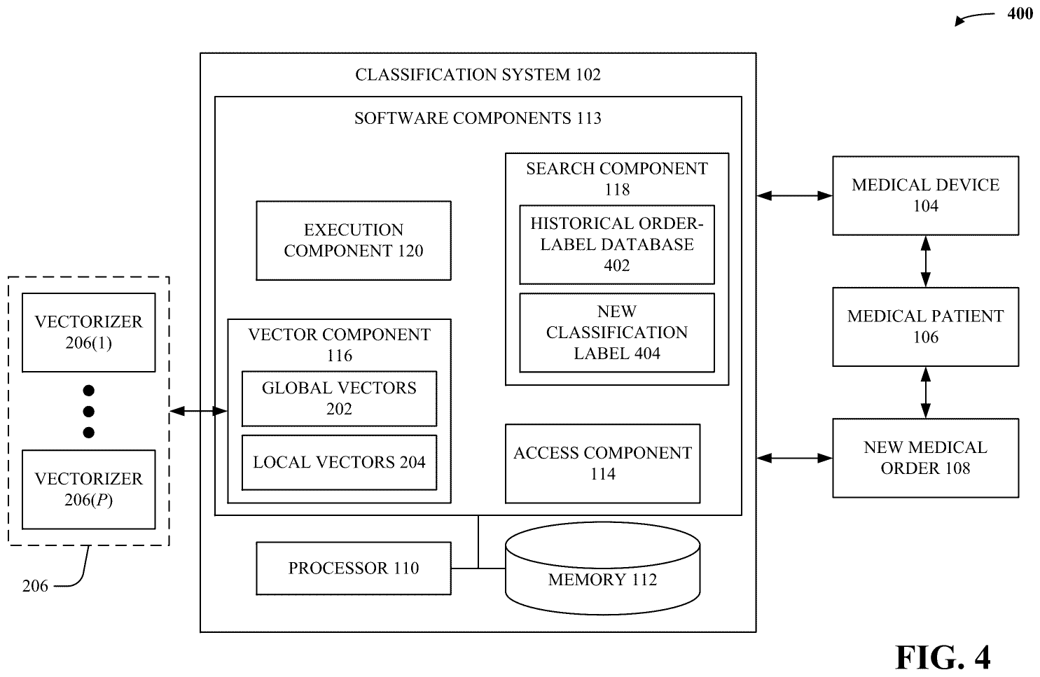

FIG. 4 illustrates a block diagram of an example, non-limiting system 400 including an historical order-label database and a new classification label that can facilitate global and local search-based classification of text in accordance with one or more embodiments described herein. As shown, the system 400 can, in some cases, comprise the same components as the system 200, and can further comprise an historical order-label database 402 and a new classification label 404.

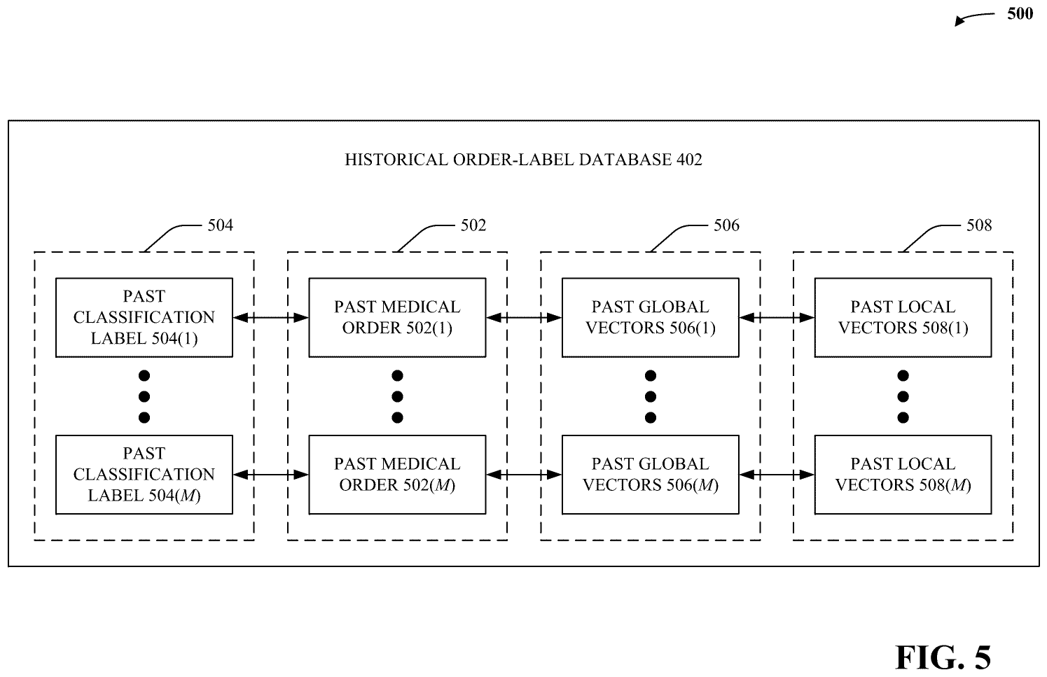

In various embodiments, the search component 118 can electronically store, electronically maintain, electronically control, or otherwise electronically access the historical order-label database 402. In various aspects, the search component 118 can assign to the new medical order 108 the new classification label 404, by searching through the historical order-label database 402 using both the one or more global vectors 202 and the set of local vectors 204. Non-limiting aspects are described with respect to FIGS. 5-9.

FIGS. 5-9 illustrate example, non-limiting block diagrams showing how the new classification label 404 can be identified by searching the historical order-label database 402 with the one or more global vectors 202 and with the set of local vectors 204 in accordance with one or more embodiments described herein.

First, consider FIG. 5. FIG. 5 shows a block diagram 500 of a non-limiting example of the historical order-label database 402.

As shown, the historical order-label database 402 can comprise a plurality of past medical orders 502. In various aspects, the plurality of past medical orders 502 can comprise m orders, for any suitable positive integer m>1: a past medical order 502(1) to a past medical order 502(m). In various instances, each of the plurality of past medical orders 502 can have the same format or dimensionality as the new medical order 108. Accordingly, each of the plurality of past medical orders 502 can be any suitable electronic document comprising n textual sections that textually orders, requests, calls for, or otherwise prescribes the performance of any suitable medical action on or with respect to some respective past medical patient. Note that different ones of the plurality of past medical orders 502 can come or originate from the same or different medical sites as each other. Accordingly, the language, jargon, or phraseology used in any one of the plurality of past medical orders 502 can be the same or different than the language or phraseology used in any other one of the plurality of past medical orders 502.

In various cases, as shown, the historical order-label database 402 can comprise a plurality of past classification labels 504. In various aspects, the plurality of past classification labels 504 can respectively correspond to the plurality of past medical orders 502. Thus, since the plurality of past medical orders 502 can comprise m orders, the plurality of past classification labels 504 can comprise m labels: a past classification label 504(1) to a past classification label 504(m). In various instances, each of the plurality of past classification labels 504(1) can be any suitable electronic data (e.g., one or more scalars, one or more vectors, one or more matrices, one or more tensors, one or more character strings, or any suitable combination thereof) that specifies or indicates which specific medical action is requested, called for, or prescribed by a respective one of the plurality of past medical orders 502. As a non-limiting example, the past classification label 504(1) can correspond to the past medical order 502(1). Thus, the past classification label 504(1) can specify, indicate, or otherwise identify whatever specific medical action (e.g., imaging protocol, medication dosage, surgical procedure) that is known or deemed to be called for or prescribed by the past medical order 502(1). As another non-limiting example, the past classification label 504(m) can correspond to the past medical order 502(m). So, the past classification label 504(m) can specify, indicate, or otherwise identify whatever specific medical action that is known or deemed to be called for or prescribed by the past medical order 502(m).

In various aspects, as shown, the historical order-label database 402 can comprise a plurality of past global vectors 506. In various instances, the plurality of past global vectors 506 can respectively correspond to the plurality of past medical orders 502. As a non-limiting example, the plurality of past global vectors 506 can comprise one or more past global vectors 506(1) that correspond to the past medical order 502(1). In various cases, the vector component 116 can compute the one or more past global vectors 506(1) by applying or executing the one or more vectorizers 206 to or on the past medical order 502(1) as described with respect to FIG. 3. As another non-limiting example, the plurality of past global vectors 506 can comprise one or more past global vectors 506(m) that correspond to the past medical order 502(m). As above, the vector component 116 can compute the one or more past global vectors 506(m) by applying or executing the one or more vectorizers 206 to or on the past medical order 502(m) as described with respect to FIG. 3. In various cases, the one or more past global vectors 506(1) to the one or more past global vectors 506(m) can collectively be considered as the plurality of past global vectors 506.

In various aspects, as shown, the historical order-label database 402 can comprise a plurality of past local vectors 508. In various instances, the plurality of past local vectors 508 can respectively correspond to the plurality of past medical orders 502. As a non-limiting example, the plurality of past local vectors 508 can comprise a set of past local vectors 508(1) that correspond to the past medical order 502(1). In various cases, the vector component 116 can compute the set of past local vectors 508(1) by applying or executing the one or more vectorizers 206 to or on the past medical order 502(1) as described with respect to FIG. 3. As another non-limiting example, the plurality of past local vectors 508 can comprise the set of past local vectors 508(m) that correspond to the past medical order 502(m). As above, the vector component 116 can compute the set of past local vectors 508(m) by applying or executing the one or more vectorizers 206 to or on the past medical order 502(m) as described with respect to FIG. 3. In various cases, the set of past local vectors 508(1) to the set of past local vectors 508(m) can collectively be considered as the plurality of past local vectors 508.

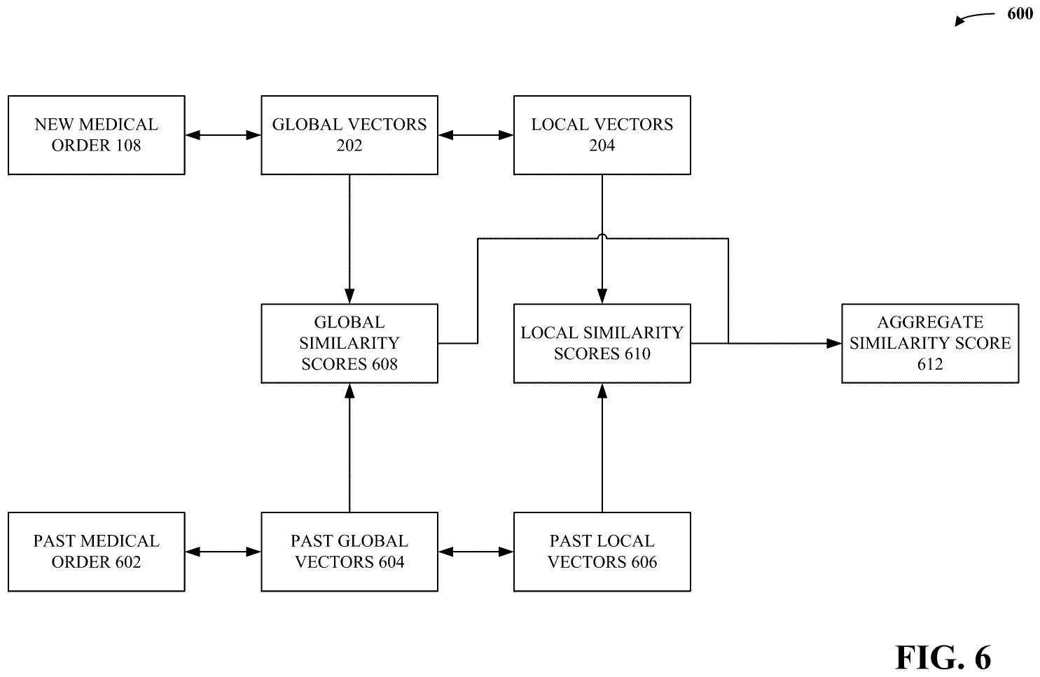

Now, consider FIG. 6. In various embodiments, as mentioned above, the vector component 116 can compute the one or more global vectors 202 and the set of local vectors 204 for the new medical order 108. In various aspects, as shown in the non-limiting example block diagram 600 of FIG. 6, there can be a past medical order 602, which can be any of the plurality of past medical orders 502. In various instances, one or more past global vectors 604 can be whichever of the plurality of past global vectors 506 correspond to the past medical order 602. Likewise, a set of past local vectors 606 can be whichever of the plurality of past local vectors 508 correspond to the past medical order 602.

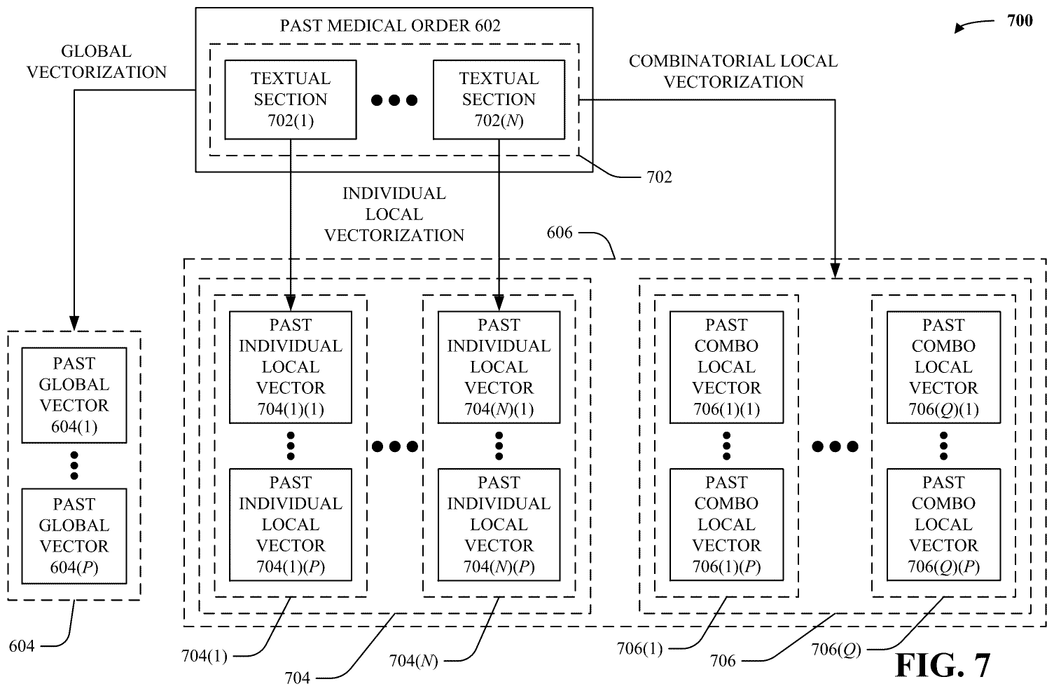

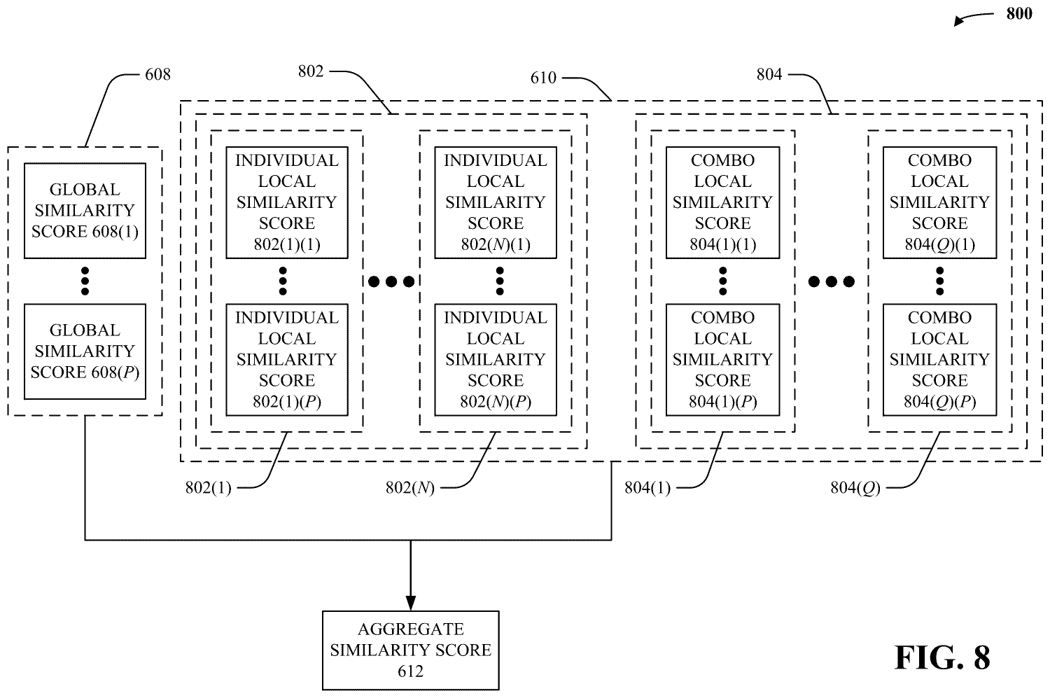

In various cases, the search component 118 can respectively compare the one or more global vectors 202 to the one or more past global vectors 604. Such comparison can yield one or more global similarity scores 608, which can indicate how numerically similar or dissimilar the one or more global vectors 202 are to the one or more past global vectors 604. In various aspects, the search component 118 can also respectively compare the set of local vectors 204 to the set of past local vectors 606. Such comparison can yield a set of local similarity scores 610, which can indicate how numerically similar or dissimilar the set of local vectors 204 are to the set of past local vectors 606. In various instances, the search component 118 can combine the one or more global similarity scores 608 with the set of local similarity scores 610 in any suitable fashion, thereby yielding an aggregate similarity score 612. In various cases, the aggregate similarity score 612 can be considered as indicating how semantically similar or dissimilar the past medical order 602 is to the new medical order 108. Non-limiting aspects are described with respect to FIGS. 7-8.

Consider FIG. 7. As shown in the non-limiting example block diagram 700 of FIG. 7, the past medical order 602 can comprise a plurality of textual sections 702. In various aspects, the plurality of textual sections 702 can have the same cardinality as the plurality of textual sections 302. Thus, since the plurality of textual sections 302 comprises n sections, the plurality of textual sections 702 can likewise comprise n sections: a textual section 702(1) to a textual section 702(n). In various instances, since medical orders can have standardized formats, each of the plurality of textual sections 702 can be of a same type as a respective one of the plurality of textual sections 302. As a non-limiting example, suppose that the textual section 302(1) is a “patient symptoms” text field of the new medical order 108. In such case, the textual section 702(1) can likewise be a “patient symptoms” text field of the past medical order 602. As another non-limiting example, suppose that the textual section 302(m) is a “doctor observations” text field of the new medical order 108. In such case, the textual section 702(m) can likewise be a “doctor observations” text field of the past medical order 602.

In various embodiments, the vector component 116 can electronically generate the one or more past global vectors 604, by applying or executing the one or more vectorizers 206 to or on the past medical order 602 in its entirety.

As a non-limiting example, the vector component 116 can electronically apply or execute the vectorizer 206(1) on the entirety of the past medical order 602, thereby computing, producing, or yielding a past global vector 604(1). As above, the past global vector 604(1) can, despite its name, be one or more scalars, one or more vectors, one or more matrices, one or more tensors, or any suitable combination thereof that numerically represent any suitable semantic characteristics or properties (e.g., those which the vectorizer 206(1) can capture) of the entirety of the past medical order 602.

As another non-limiting example, the vector component 116 can electronically apply or execute the vectorizer 206(p) on the entirety of the past medical order 602, thereby computing, producing, or yielding a past global vector 604(p). As above, the past global vector 604(p) can, despite its name, be one or more scalars, one or more vectors, one or more matrices, one or more tensors, or any suitable combination thereof that numerically represent any suitable semantic characteristics or properties (e.g., those which the vectorizer 206(p) can capture) of the entirety of the past medical order 602.

In various aspects, the past global vector 604(1) to the past global vector 604(p) can collectively be considered as the one or more past global vectors 604. Moreover, in various instances, each of the one or more past global vectors 604 can have the same dimensionality as the one or more global vectors 202.

Moving on, in various embodiments, the vector component 116 can electronically generate the set of past local vectors 606, by applying or executing the one or more vectorizers 206 to or on the past medical order 602 not in its entirety.

Indeed, in some cases, the vector component 116 can electronically apply or execute the one or more vectorizers 206 individually on each of the plurality of textual sections 702, thereby yielding a set of past individual local vectors 704.

As a non-limiting example, the vector component 116 can electronically apply or execute the vectorizer 206(1) on the textual section 702(1) alone, thereby computing, producing, or yielding a past individual local vector 704(1)(1). In various aspects, the past individual local vector 704(1)(1) can, despite its name, be one or more scalars, one or more vectors, one or more matrices, one or more tensors, or any suitable combination thereof that numerically represent any suitable semantic characteristics or properties of the textual section 702(1) (e.g., and not of the remainder of the past medical order 602).

As another non-limiting example, the vector component 116 can electronically apply or execute the vectorizer 206(p) on the textual section 702(1) alone, thereby computing, producing, or yielding a past individual local vector 704(1)(p). In various aspects, the past individual local vector 704(1)(p) can, despite its name, be one or more scalars, one or more vectors, one or more matrices, one or more tensors, or any suitable combination thereof that numerically represent any suitable semantic characteristics or properties of the textual section 702(1).

In various aspects, the past individual local vector 704(1)(1) to the past individual local vector 704(1)(p) can collectively be considered as one or more past individual local vectors 704(1). In various instances, each of the one or more past individual local vectors 704(1) can have the same dimensionality as each other (and thus as the one or more individual local vectors 304(1)).

As yet another non-limiting example, the vector component 116 can electronically apply or execute the vectorizer 206(1) on the textual section 702(n) alone, thereby computing, producing, or yielding a past individual local vector 704(n)(1). In various aspects, the past individual local vector 704(n)(1) can, despite its name, be one or more scalars, one or more vectors, one or more matrices, one or more tensors, or any suitable combination thereof that numerically represent any suitable semantic characteristics or properties of the textual section 702(n).

As even another non-limiting example, the vector component 116 can electronically apply or execute the vectorizer 206(p) on the textual section 702(n) alone, thereby computing, producing, or yielding a past individual local vector 704(n)(p). In various aspects, the past individual local vector 704(n)(p) can, despite its name, be one or more scalars, one or more vectors, one or more matrices, one or more tensors, or any suitable combination thereof that numerically represent any suitable semantic characteristics or properties of the textual section 702(n).

In various aspects, the past individual local vector 704(n)(1) to the past individual local vector 704(n)(p) can collectively be considered as one or more past individual local vectors 704(n). In various instances, each of the one or more past individual local vectors 704(n) can have the same dimensionality as each other (and thus as the one or more individual local vectors 304(n)).

In various cases, the one or more past individual local vectors 704(1) to the one or more past individual local vectors 704(n) can collectively be considered as the set of past individual local vectors 704.

Now, as mentioned above, q distinct textual section combinations of the plurality of textual sections 302 can be selected or chosen by the vector component 116. In various embodiments, those same q textual section combinations can be selected or chosen from the plurality of textual sections 702. Accordingly, the vector component 116 can electronically apply or execute the one or more vectorizers 206 on each of such q textual section combinations, which can yield a set of past combo local vectors 706.

As a non-limiting example, the vector component 116 can electronically apply or execute the vectorizer 206(1) on the first textual section combination of the plurality of textual sections 702 alone (e.g., omitting whatever portions of the past medical order 602 are not in that first textual section combination). In various cases, such application or execution can cause the vectorizer 206(1) to compute, produce, or otherwise yield a past combo local vector 706(1)(1), which can, despite its name, be one or more scalars, one or more vectors, one or more matrices, one or more tensors, or any suitable combination thereof that numerically represent any suitable semantic characteristics or properties of that first textual section combination.

As another non-limiting example, the vector component 116 can electronically apply or execute the vectorizer 206(p) on that first textual section combination of the plurality of textual sections 702 alone, thereby computing, producing, or yielding a past combo local vector 706(1)(p). In various cases, the past combo local vector 706(1)(p) can, despite its name, be one or more scalars, one or more vectors, one or more matrices, one or more tensors, or any suitable combination thereof that numerically represent any suitable semantic characteristics or properties of that first textual section combination.

In various aspects, the past combo local vector 706(1)(1) to the past combo local vector 706(1)(p) can collectively be considered as one or more past combo local vectors 706(1). In various instances, each of the one or more past combo local vectors 706(1) can have the same dimensionality as each other (and thus as the one or more combo local vectors 306(1)).

As still another non-limiting example, the vector component 116 can electronically apply or execute the vectorizer 206(1) on the q-th textual section combination of the plurality of textual sections 702 alone (e.g., omitting whatever portions of the past medical order 602 are not in that q-th textual section combination). In various cases, such application or execution can cause the vectorizer 206(1) to compute, produce, or otherwise yield a past combo local vector 706(q)(1), which can, despite its name, be one or more scalars, one or more vectors, one or more matrices, one or more tensors, or any suitable combination thereof that numerically represent any suitable semantic characteristics or properties of that q-th textual section combination.

As even another non-limiting example, the vector component 116 can electronically apply or execute the vectorizer 206(p) on that q-th textual section combination of the plurality of textual sections 702 alone, thereby computing, producing, or yielding a past combo local vector 706(q)(p). In various cases, the past combo local vector 706(q)(p) can, despite its name, be one or more scalars, one or more vectors, one or more matrices, one or more tensors, or any suitable combination thereof that numerically represent any suitable semantic characteristics or properties of that q-th textual section combination.

In various aspects, the past combo local vector 706(q)(1) to the past combo local vector 706(q)(p) can collectively be considered as one or more past combo local vectors 706(q). In various instances, each of the one or more past combo local vectors 706(q) can have the same dimensionality as each other (and thus as the one or more combo local vectors 306(q)).

In various cases, the one or more past combo local vectors 706(1) to the one or more past combo local vectors 706(q) can collectively be considered as the set of past combo local vectors 706. Furthermore, in various aspects, the set of past individual local vectors 704 and the set of past combo local vectors 706 can collectively be considered as forming the set of past local vectors 606.

So, the one or more past global vectors 604 and the set of past local vectors 606 can together be considered as collectively capturing, encapsulating, or otherwise representing various semantic characteristics or properties of the past medical order 602 from various different scopes, scales, or levels of granularity, so as to richly or deeply encompass the semantic content of the past medical order 602.

In situations where the vector component 116 applies compression or reduction (e.g., PCA) to the one or more global vectors 202 or to the set of local vectors 204, the vector component 116 can likewise apply such compression or reduction to the one or more past global vectors 604 or to the set of past local vectors 606.

Now consider FIG. 8. As shown, the non-limiting example block diagram 800 of FIG. 8 pertains to similarity score computation.

In various embodiments, as mentioned above, the search component 118 can electronically compute the one or more global similarity scores 608, by respectively comparing the one or more global vectors 202 to the one or more past global vectors 604.

As a non-limiting example, the search component 118 can compute or calculate a global similarity score 608(1), by comparing the global vector 202(1) to the past global vector 604(1). In some cases, the global similarity score 608(1) can be equal to or otherwise based on a cosine similarity computed between the global vector 202(1) and the past global vector 604(1). In other cases, the global similarity score 608(1) can be equal to a reciprocal of, or can be otherwise based on, a Euclidean distance computed between the global vector 202(1) and the past global vector 604(1). In any case, the global similarity score 608(1) can be a real-valued scalar whose magnitude indicates how similar or dissimilar the global vector 202(1) is to the past global vector 604(1) (e.g., magnitudes that are larger can indicate more similarity; magnitudes that are smaller or closer to zero can indicate less similarity). Accordingly, the global similarity score 608(1) can be considered as indicating how similar or dissimilar the following are: whatever semantic characteristics of the entirety of the new medical order 108 that are capturable by the vectorizer 206(1); and whatever semantic characteristics of the entirety of the past medical order 602 that are capturable by the vectorizer 206(1).

As another non-limiting example, the search component 118 can compute or calculate a global similarity score 608(p), by comparing (e.g., via cosine similarity or Euclidean distance) the global vector 202(p) to the past global vector 604(p). So, the global similarity score 608(p) can be a real-valued scalar whose magnitude indicates how similar or dissimilar the global vector 202(p) is to the past global vector 604(p). Accordingly, the global similarity score 608(p) can be considered as indicating how similar or dissimilar the following are: whatever semantic characteristics of the entirety of the new medical order 108 that are capturable by the vectorizer 206(p); and whatever semantic characteristics of the entirety of the past medical order 602 that are capturable by the vectorizer 206(p).

In various cases, the global similarity score 608(1) to the global similarity score 608(p) can collectively be considered as the one or more global similarity scores 608.

Moving on, the search component 118 can, as mentioned above, electronically compute the set of local similarity scores 610, by respectively comparing the set of local vectors 204 to the set of past local vectors 606.

As a non-limiting example, the search component 118 can compute or calculate an individual local similarity score 802(1)(1), by comparing (e.g., via cosine similarity or Euclidean distance) the individual local vector 304(1)(1) to the past individual local vector 704(1)(1). Thus, the individual local similarity score 802(1)(1) can be a real-valued scalar whose magnitude indicates how similar or dissimilar the individual local vector 304(1)(1) is to the past individual local vector 704(1)(1). Accordingly, the individual local similarity score 802(1)(1) can be considered as indicating how similar or dissimilar the following are: whatever semantic characteristics of the textual section 302(1) that are capturable by the vectorizer 206(1); and whatever semantic characteristics of the textual section 702(1) that are capturable by the vectorizer 206(1).

As another non-limiting example, the search component 118 can compute or calculate an individual local similarity score 802(1)(p), by comparing (e.g., via cosine similarity or Euclidean distance) the individual local vector 304(1)(p) to the past individual local vector 704(1)(p). So, the individual local similarity score 802(1)(p) can be a real-valued scalar whose magnitude indicates how similar or dissimilar the individual local vector 304(1)(p) is to the past individual local vector 704(1)(p). Thus, the individual local similarity score 802(1)(p) can be considered as indicating how similar or dissimilar the following are: whatever semantic characteristics of the textual section 302(1) that are capturable by the vectorizer 206(p); and whatever semantic characteristics of the textual section 702(1) that are capturable by the vectorizer 206(p).

In various cases, the individual local similarity score 802(1)(1) to the individual local similarity score 802(1)(p) can collectively be considered as one or more individual local similarity scores 802(1).

As still another non-limiting example, the search component 118 can compute or calculate an individual local similarity score 802(n)(1), by comparing (e.g., via cosine similarity or Euclidean distance) the individual local vector 304(n)(1) to the past individual local vector 704(n)(1). Thus, the individual local similarity score 802(n)(1) can be a real-valued scalar whose magnitude indicates how similar or dissimilar the individual local vector 304(n)(1) is to the past individual local vector 704(n)(1). Accordingly, the individual local similarity score 802(n)(1) can be considered as indicating how similar or dissimilar the following are: whatever semantic characteristics of the textual section 302(n) that are capturable by the vectorizer 206(1); and whatever semantic characteristics of the textual section 702(n) that are capturable by the vectorizer 206(1).

As another non-limiting example, the search component 118 can compute or calculate an individual local similarity score 802(n)(p), by comparing (e.g., via cosine similarity or Euclidean distance) the individual local vector 304(n)(p) to the past individual local vector 704(n)(p). So, the individual local similarity score 802(n)(p) can be a real-valued scalar whose magnitude indicates how similar or dissimilar the individual local vector 304(n)(p) is to the past individual local vector 704(n)(p). Thus, the individual local similarity score 802(n)(p) can be considered as indicating how similar or dissimilar the following are: whatever semantic characteristics of the textual section 302(n) that are capturable by the vectorizer 206(p); and whatever semantic characteristics of the textual section 702(n) that are capturable by the vectorizer 206(p).

In various cases, the individual local similarity score 802(n)(1) to the individual local similarity score 802(n)(p) can collectively be considered as one or more individual local similarity scores 802(n).

In various aspects, the one or more individual local similarity scores 802(1) to the one or more individual local similarity scores 802(n) can collectively be considered as a set of individual local similarity scores 802.

As another non-limiting example, the search component 118 can compute or calculate a combo local similarity score 804(1)(1), by comparing (e.g., via cosine similarity or Euclidean distance) the combo local vector 306(1)(1) to the past combo local vector 706(1)(1). Thus, the combo local similarity score 804(1)(1) can be a real-valued scalar whose magnitude indicates how similar or dissimilar the combo local vector 306(1)(1) is to the past combo local vector 706(1)(1). Accordingly, the combo local similarity score 804(1)(1) can be considered as indicating how similar or dissimilar the following are: whatever semantic characteristics of the first combination of the plurality of textual sections 302 that are capturable by the vectorizer 206(1); and whatever semantic characteristics of the first combination of the plurality of textual sections 702 that are capturable by the vectorizer 206(1).

As yet another non-limiting example, the search component 118 can compute or calculate a combo local similarity score 804(1)(p), by comparing (e.g., via cosine similarity or Euclidean distance) the combo local vector 306(1)(p) to the past combo local vector 706(1)(p). Thus, the combo local similarity score 804(1)(p) can be a real-valued scalar whose magnitude indicates how similar or dissimilar the combo local vector 306(1)(p) is to the past combo local vector 706(1)(p). In other words, the combo local similarity score 804(1)(p) can be considered as indicating how similar or dissimilar the following are: whatever semantic characteristics of the first combination of the plurality of textual sections 302 that are capturable by the vectorizer 206(p); and whatever semantic characteristics of the first combination of the plurality of textual sections 702 that are capturable by the vectorizer 206(p).

In various cases, the combo local similarity score 804(1)(1) to the combo local similarity score 804(1)(p) can collectively be considered as one or more combo local similarity scores 804(1).

As still another non-limiting example, the search component 118 can compute or calculate a combo local similarity score 804(q)(1), by comparing (e.g., via cosine similarity or Euclidean distance) the combo local vector 306(q)(1) to the past combo local vector 706(q)(1). Thus, the combo local similarity score 804(q)(1) can be a real-valued scalar whose magnitude indicates how similar or dissimilar the combo local vector 306(q)(1) is to the past combo local vector 706(q)(1). Accordingly, the combo local similarity score 804(q)(1) can be considered as indicating how similar or dissimilar the following are: whatever semantic characteristics of the q-th combination of the plurality of textual sections 302 that are capturable by the vectorizer 206(1); and whatever semantic characteristics of the q-th combination of the plurality of textual sections 702 that are capturable by the vectorizer 206(1).

As even another non-limiting example, the search component 118 can compute or calculate a combo local similarity score 804(q)(p), by comparing (e.g., via cosine similarity or Euclidean distance) the combo local vector 306(q)(p) to the past combo local vector 706(q)(p). So, the combo local similarity score 804(q)(p) can be a real-valued scalar whose magnitude indicates how similar or dissimilar the combo local vector 306(q)(p) is to the past combo local vector 706(q)(p). In other words, the combo local similarity score 804(q)(p) can be considered as indicating how similar or dissimilar the following are: whatever semantic characteristics of the q-th combination of the plurality of textual sections 302 that are capturable by the vectorizer 206(p); and whatever semantic characteristics of the q-th combination of the plurality of textual sections 702 that are capturable by the vectorizer 206(p).

In various cases, the combo local similarity score 804(q)(1) to the combo local similarity score 804(q)(p) can collectively be considered as one or more combo local similarity scores 804(q).

In various instances, the one or more combo local similarity scores 804(1) to the one or more combo local similarity scores 804(q) can collectively be considered as a set of combo local similarity scores 804.

In various aspects, the set of individual local similarity scores 802 and the set of combo local similarity scores 804 can collectively be considered as forming the set of local similarity scores 610.

Now, in various embodiments, the search component 118 can electronically combine or aggregate the one or more global similarity scores 608 and the set of local similarity scores 610, thereby forming the aggregate similarity score 612. As a non-limiting example, the aggregate similarity score 612 can be equal to or otherwise based on an unweighted average of the one or more global similarity scores 608 and the set of local similarity scores 610. As another non-limiting example, the aggregate similarity score 612 can be equal to or otherwise based on a weighted average of the one or more global similarity scores 608 and the set of local similarity scores 610. For instance, the following can be implemented in some cases: a global weighting coefficient can be multiplicatively applied to the one or more global similarity scores 608; a first individual weighting coefficient can be multiplicatively applied to the one or more individual local similarity scores 802(1); an n-th individual weighting coefficient can be multiplicatively applied to the one or more individual local similarity scores 802(n); a first combo weighting coefficient can be multiplicatively applied to the one or more combo local similarity scores 804(1); and a q-th combo weighting coefficient can be multiplicatively applied to the one or more combo local similarity scores 804(q). As another instance, the following can be implemented in some cases: a first weighting coefficient can be multiplicatively applied to every similarity score that was derived from vectors produced by the vectorizer 206(1); and a p-th weighting coefficient can be multiplicatively applied to every similarity score that was derived from vectors produced by the vectorizer 206(p). In any case, the aggregate similarity score 612 can be a real-valued scalar whose magnitude indicates how similarity or dissimilar the overall semantic properties of the past medical order 602 are to those of the new medical order 108.

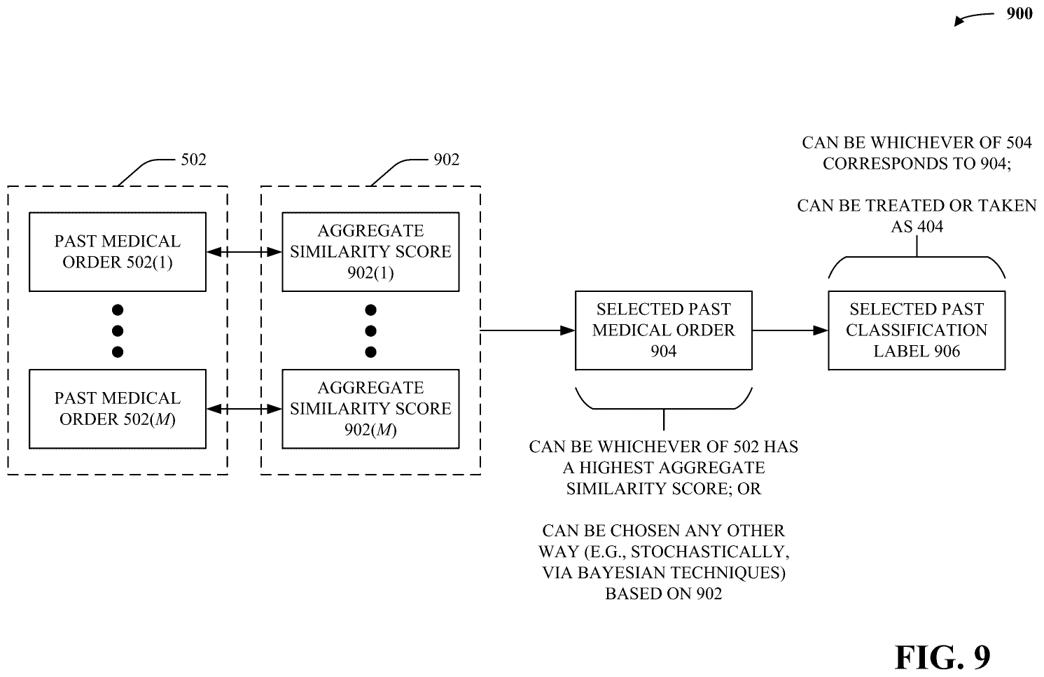

Next, consider the non-limiting example block diagram 900 of FIG. 9. In various embodiments, the search component 118 can electronically compute (as described with respect to FIGS. 6-8) an aggregate similarity score for each of the plurality of past medical orders 502. This can yield a plurality of aggregate similarity scores 902 that respectively correspond to the plurality of past medical orders 502 (e.g., an aggregate similarity score 902(1) can indicate how similar or dissimilar the past medical order 502(1) is to the new medical order 108; an aggregate similarity score 902(m) can indicate how similar or dissimilar the past medical order 502(m) is to the new medical order 108). In various aspects, the search component 118 can electronically select or choose any of the plurality of past medical orders 502 that are sufficiently or adequately similar to the new medical order 108, by consulting the plurality of aggregate similarity scores 902. Such selected or chosen past medical order can be referred to as a selected past medical order 904.

As a non-limiting example, the selected past medical order 904 can be whichever of the plurality of past medical orders 502 is most semantically similar to the new medical order 108 (e.g., can be whichever of 502 has a highest or largest aggregate similarity score).

As another non-limiting example, the selected past medical order 904 can be chosen from the plurality of past medical orders 502 stochastically (e.g., if multiple past medical orders have an aggregate similarity score that exceeds any suitable threshold value, then the selected past medical order 904 can be chosen randomly from those multiple past medical orders).

As yet another non-limiting example, the selected past medical order 904 can be chosen by applying any suitable Bayesian technique or selection algorithm, such as BordaCount, to the plurality of aggregate similarity scores 902.

As even another non-limiting example, the selected past medical order 904 can be chosen by applying any graph selection technique or clustering techniques to the plurality of aggregate similarity scores 902.

Although FIG. 8 shows a distinct similarity score being computed for each corresponding pair of global vectors, for each corresponding pair of individual local vectors, and for each corresponding pair of combo local vectors between the new medical order 108 and the past medical order 602, and although FIG. 9 shows a distinct aggregate similarity score being computed for each of the plurality of past medical orders 502, this is a mere non-limiting example for ease of explanation and illustration. It should be appreciated that, in various embodiments, the search component 118 can implement, utilize, or otherwise leverage any suitable searching optimizations or heuristics, so as to reduce the total number of similarity scores that are computed or so as to otherwise increase searching efficiency. As a non-limiting example, the search component 118 can leverage FAISS for comparing the global vectors, the individual local vectors, and the combo local vectors between any given past medical order and the new medical order 108.

Furthermore, although the herein disclosure has mainly described various embodiments in which the search component 118 computes and aggregates similarity scores, these are mere non-limiting examples for ease of explanation and illustration. It should be appreciated that the search component 118 can compute ranks instead of, or possibly as a supplement to, any of the herein described similarity scores. As a non-limiting example, the search component 118 can rank the plurality of past medical orders 502 in terms of embedding similarity to the new medical order 108. In some instances, the search component 118 can granularly rank the global vectors, the individual local vectors, or the combo local vectors of the plurality of past medical orders 502 in terms of embedding similarity to the global vectors, individual local vectors, or combo local vectors of the new medical order 108 (e.g., a given past medical order can have a total of p global vectors, np individual local vectors, and qp combo local vectors; so, the search component 118 can compute a total of (1+n+q) p granular rankings for the given past medical order). The search component 118 can accordingly aggregate (e.g., via weighted or unweighted averaging, via BordaCount, via any suitable graph technique) those granular rankings into overall rankings showing which of the plurality of past medical orders 502 are most or least similar to the new medical order 108. In such situations, the selected past medical order 904 can be whichever of the plurality of past medical orders 502 has a highest overall ranking with respect to the new medical order 108.

In any case, the selected past medical order 904 can be considered as having at least a threshold amount of semantic similarity to the new medical order 108.

In various aspects, the search component 118 can identify whichever of the plurality of past classification labels 504 that corresponds to the selected past medical order 904. In various instances, such past classification label can be referred to as a selected past classification label 906. Note that the selected past classification label 906 can be considered as indicating, identifying, or specifying whatever specific medical action is requested, called for, or prescribed by the selected past medical order 904. In various cases, the new classification label 404 can be created so as to be equal to or the same as the selected past classification label 906. After all, since the new medical order 108 can have a threshold amount of semantic similarity to the selected past medical order 904, it can be expected that the new medical order 108 and the selected past medical order 904 should have the same classification label as each other. In other words, since the new medical order 108 can have a threshold amount of semantic similarity to the selected past medical order 904, it can be expected that they request, call for, or prescribe the same medical action as each other.

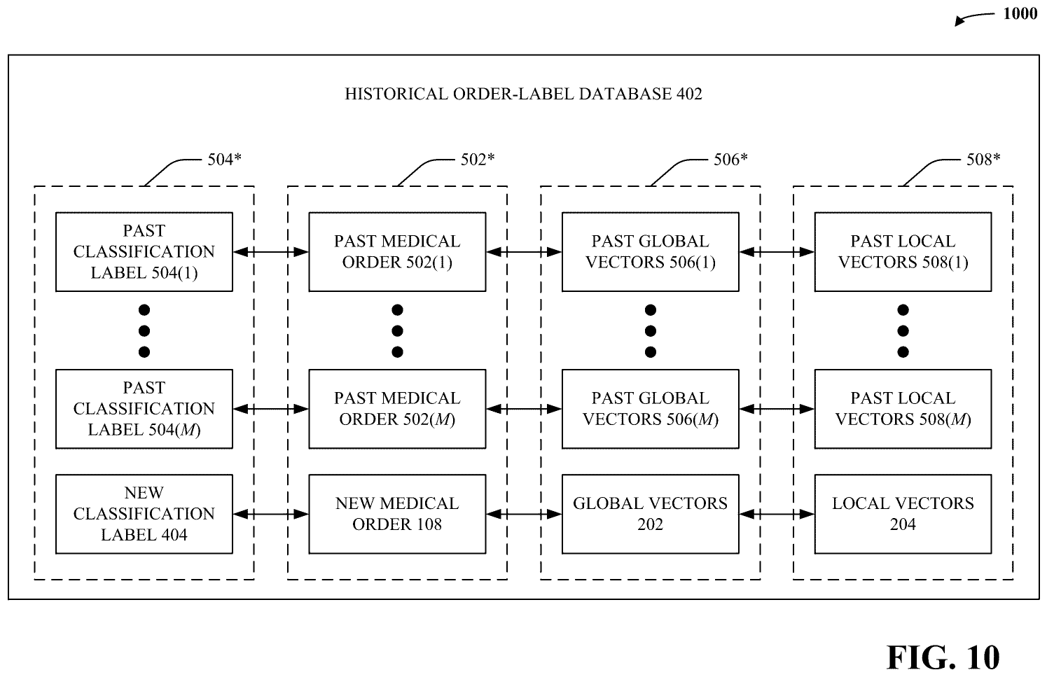

Now, consider the non-limiting example block diagram 1000 of FIG. 10. In various embodiments, the search component 118 can electronically edit or update the historical order-label database 402, based on the new medical order 108 and the new classification label 404. More specifically, the search component 118 can insert as a new entry the new medical order 108, the new classification label 404, the one or more global vectors 202, and the set of local vectors 204 into the historical order-label database 402, thereby expanding the historical order-label database 402. For instance, the search component 118 can insert the new medical order 108 into the plurality of past medical orders 502, thereby yielding an edited or enlarged plurality of past medical orders 502*. Likewise, the search component 118 can insert the new classification label 404 into the plurality of past classification labels 504, thereby yielding an edited or enlarged plurality of past classification labels 504*. Similarly, the search component 118 can insert the one or more global vectors 202 into the plurality of past global vectors 506, thereby yielding an edited or enlarged plurality of past global vectors 506*. Furthermore, the search component 118 can insert the set of local vectors 204 into the plurality of past local vectors 508, thereby yielding an edited or enlarged plurality of past local vectors 508*. Note that such insertions can enable future medical orders to be compared semantically against the new medical order 108. In some cases, such insertions can be conditioned or predicated upon prior user approval, verification, or confirmation that the new classification label 404 is appropriate for the new medical order 108. In other words, such insertions can be considered as keeping the historical order-label database 402 up-to-date.

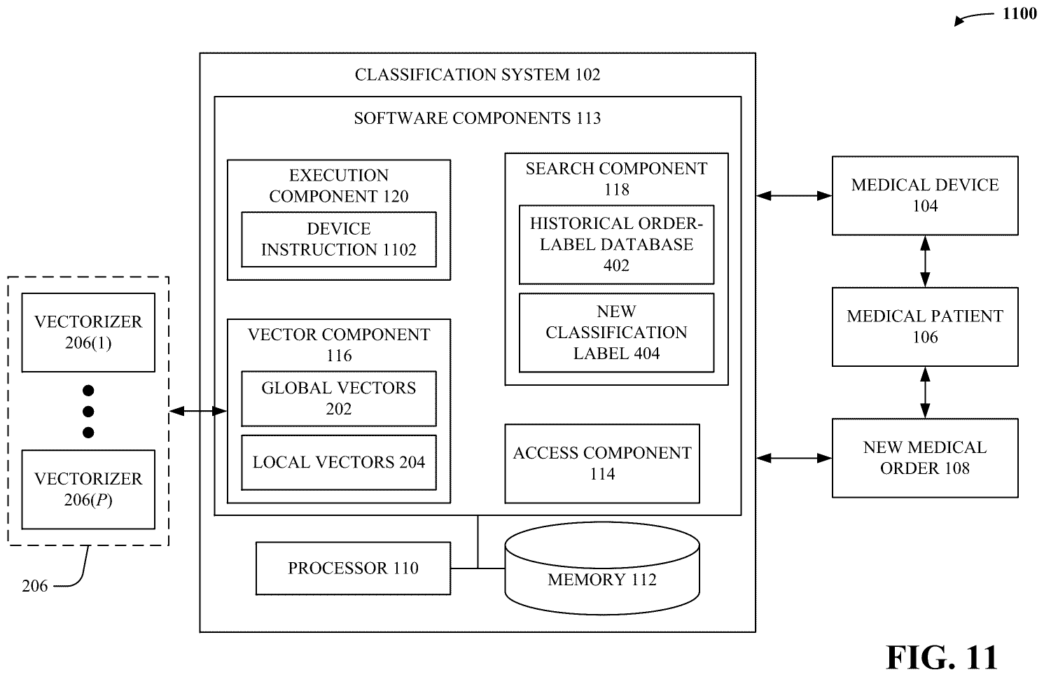

FIG. 11 illustrates a block diagram of an example, non-limiting system 1100 including a device instruction that can facilitate global and local search-based classification of text in accordance with one or more embodiments described herein. As shown, the system 1100 can, in some cases, comprise the same components as the system 400, and can further comprise a device instruction 1102.

In various embodiments, as mentioned above, the new classification label 404 can indicate, specify, or identify what specific medical action is requested, called for, or prescribed by the new medical order 108. In various aspects, also as mentioned above, that specific medical action can be automatically performable by the medical device 104. Accordingly, in various instances, the execution component 120 can electronically transmit the device instruction 1102 to the medical device 104, which can cause the medical device 104 to automatically perform, initiate, or otherwise activate whatever specific medical action is indicated by the new classification label 404.

As a non-limiting example, suppose that the medical device 104 is a medical imaging scanner in which the medical patient 106 is positioned, and suppose that the specific medical action indicated by the new classification label 404 is a particular scanning protocol for that medical imaging scanner (e.g., the particular scanning protocol scan specify operational settings to be used by that medical imaging scanner, such as: gantry speed; gantry angular range; or scanner radiation level). Thus, the device instruction 1102 can cause the medical device 104 to automatically scan the medical patient 106 in accordance with that particular scanning protocol.

As another non-limiting example, suppose that the medical device 104 is an automated tank or reservoir containing a fluid medication and coupled via medical tubing to an airway or blood vessel of the medical patient 106, and suppose that the specific medical action indicated by the new classification label 404 is a particular medication dosage that is to be dispensed by that automated tank or reservoir (e.g., can specify how much volume or mass of the fluidic medication should be pumped into the medical patient 106). Thus, the device instruction 1102 can cause the medical device 104 to automatically pump the specified dosage of the fluidic medication into the airway or blood vessel of the medical patient 106.

As yet another non-limiting example, suppose that the medical device 104 is an automated surgery robot that is prepared to surgically operate on the medical patient 106, and suppose that the specific medical action indicated by the new classification label 404 is a particular surgical protocol to be carried out by that automated surgery robot (e.g., can specify what types of incisions or ablations to make where on or in the medical patient 106). Thus, the device instruction 1102 can cause the medical device 104 to automatically perform that particular surgical protocol on the medical patient 106.

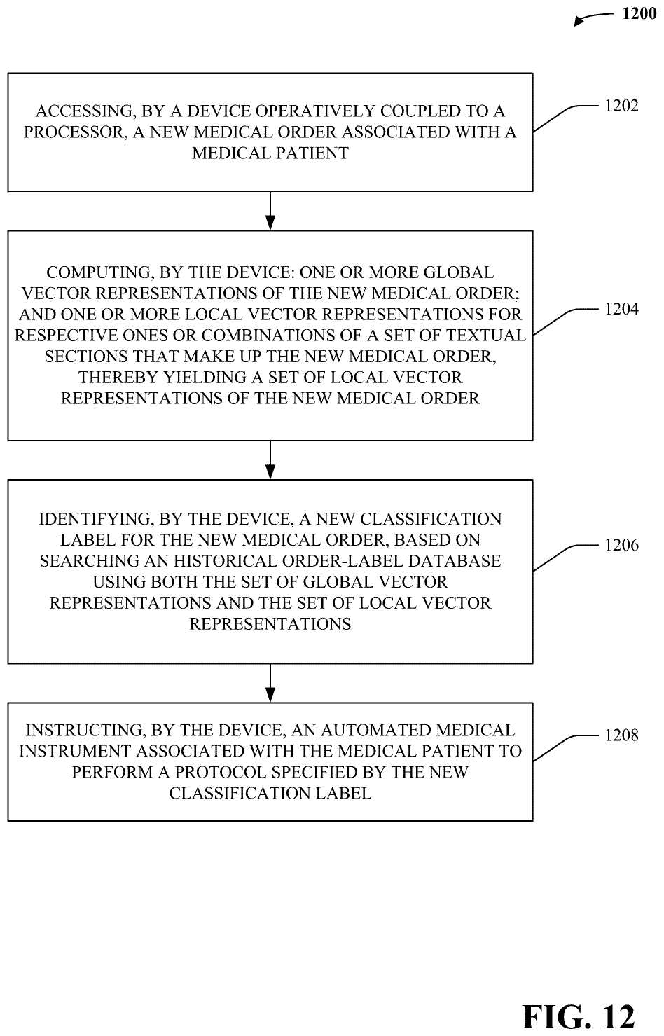

FIG. 12 illustrates a flow diagram of an example, non-limiting computer-implemented method 1200 that can facilitate global and local search-based classification of text in accordance with one or more embodiments described herein. In various cases, the classification system 102 can facilitate the computer-implemented method 1200.

In various embodiments, act 1202 can include accessing, by a device (e.g., 114) operatively coupled to a processor (e.g., 110), a new medical order (e.g., 108) associated with a medical patient (e.g., 106).

In various aspects, act 1204 can include computing, by the device (e.g., via 116): one or more global vector representations (e.g., 202) of the new medical order; and one or more local vector representations for respective ones (e.g., 304; in other words, a respective local vector per individual or single textual section) or combinations (e.g., 306; in other words, a respective local vector per distinct combination of textual sections) of a set of textual sections (e.g., 302) that make up the new medical order, thereby yielding a set of local vector representations (e.g., 204) of the new medical order.

In various instances, act 1206 can include identifying, by the device (e.g., 118), a new classification label (e.g., 404) for the new medical order, based on searching an historical order-label database (e.g., 402) using both the set of global vector representations and the set of local vector representations.

In various cases, act 1208 can include instructing, by the device (e.g., via 120), an automated medical instrument (e.g., 104) associated with the medical patient to perform a protocol specified by the new classification label.

Although not explicitly shown in FIG. 12, the device can generate the one or more global vector representations and the set of local vector representations via a term-frequency-inverse-domain-frequency vectorizer (e.g., one of 206) or via one or more encoders (e.g., one or more of 206) of one or more respective, pre-trained large language models.

Although not explicitly shown in FIG. 12, the historical order-label database can comprise: a plurality of past medical orders (e.g., 502); a plurality of past classification labels (e.g., 504) respectively corresponding to the plurality of past medical orders; one or more past global vector representations (e.g., shown in 506) for each respective one of the plurality of past medical orders; and a set of past local vector representations (e.g., shown in 508) for each respective one of the plurality of past medical orders. In various aspects, the computer-implemented method 1200 can further comprise: comparing, by the device (e.g., 118) and via cosine similarity computation or Euclidean distance computation, the one or more global vector representations of the new medical order to the one or more past global vector representations (e.g., 604) of each respective one (e.g., 602) of the plurality of past medical orders, thereby yielding one or more global similarity scores (e.g., 608) for each respective one of the plurality of past medical orders; and comparing, by the device (e.g., via 118) and via cosine similarity computation or Euclidean distance computation, the set of local vector representations of the new medical order to the set of past local vector representations (e.g., 606) of each respective one of the plurality of past medical orders, thereby yielding a set of local similarity scores (e.g., 610) for each respective one of the plurality of past medical orders.

Although not explicitly shown in FIG. 12, the computer-implemented method 1200 can further comprise: aggregating, by the device (e.g., 118) and in weighted or unweighted fashion, the one or more global similarity scores and the set of local similarity scores for each respective one of the plurality of past medical orders, thereby yielding an aggregate similarity score (e.g., 612) for each respective one of the plurality of past medical orders; and identifying, by the device (e.g., via 118), from the plurality of past medical orders, and based on the aggregate similarity scores (e.g., 902), a most-similar past medical order (e.g., 904), wherein the new classification label can be whichever (e.g., 906) of the plurality of past classification labels that corresponds to the most-similar past medical order.

Although not explicitly shown in FIG. 12, the computer-implemented method 1200 can further comprise: inserting, by the device (e.g., via 118), the new medical order, the one or more global vector representations, the set of local vector representations, and the new classification label as a new entry into the historical order-label database.

To help demonstrate various benefits of various embodiments described herein, the present inventors conducted various experiments. Such experiments involved six separate verification datasets, with each verification dataset comprising medical orders and their corresponding ground-truth classification labels.

During a first experiment, a pre-trained LLM was fine-tuned on 200 first samples from the first verification dataset and was then executed on the remainder of the first verification dataset. The fine-tuned LLM in such first experiment achieved a macro-F1 score of 86.6. Also during that first experiment, an embodiment as described herein was reduced to practice using those 200 first samples. That is, the historical order-label database 402 of such embodiment comprised a mere 200 medical orders and their corresponding labels, global vectors, and local vectors. The embodiment was then used to classify the remainder of the first verification dataset, achieving a macro-F1 score of 82.0. Thus, in the first experiment, the embodiment as described herein achieved essentially comparable performance, notwithstanding having a several-order-of-magnitude-smaller computational footprint.

During a second experiment, a pre-trained LLM was fine-tuned on 200 second samples from the second verification dataset and was then executed on the remainder of the second verification dataset. The fine-tuned LLM in such second experiment achieved a macro-F1 score of 48.6. Also during that second experiment, an embodiment as described herein was reduced to practice using those 200 second samples. That is, the historical order-label database 402 of such embodiment comprised a mere 200 medical orders and their corresponding labels, global vectors, and local vectors. The embodiment was then used to classify the remainder of the second verification dataset, achieving a macro-F1 score of 62.1. Thus, in the second experiment, the embodiment as described herein achieved significantly better performance, notwithstanding having a several-order-of-magnitude-smaller computational footprint.

During a third experiment, a pre-trained LLM was fine-tuned on 200 third samples from the third verification dataset and was then executed on the remainder of the third verification dataset. The fine-tuned LLM in such third experiment achieved a macro-F1 score of 97.7. Also during that third experiment, an embodiment as described herein was reduced to practice using those 200 third samples and was then used to classify the remainder of the third verification dataset, achieving a macro-F1 score of 97.8. Thus, in the third experiment, the embodiment as described herein achieved comparable performance, notwithstanding having a several-order-of-magnitude-smaller computational footprint.

During a fourth experiment, a pre-trained LLM was fine-tuned on 200 fourth samples from the fourth verification dataset and was then executed on the remainder of the fourth verification dataset. The fine-tuned LLM in such fourth experiment achieved a macro-F1 score of 61.4. Also during that fourth experiment, an embodiment as described herein was reduced to practice using those 200 fourth samples and was then used to classify the remainder of the fourth verification dataset, achieving a macro-F1 score of 75.5. Thus, in the fourth experiment, the embodiment as described herein achieved better performance, notwithstanding having a several-order-of-magnitude-smaller computational footprint.

During a fifth experiment, a pre-trained LLM was fine-tuned on 200 fifth samples from the fifth verification dataset and was then executed on the remainder of the fifth verification dataset. The fine-tuned LLM in such fifth experiment achieved a macro-F1 score of 84.1. Also during that fifth experiment, an embodiment as described herein was reduced to practice using those 200 fifth samples and was then used to classify the remainder of the fifth verification dataset, achieving a macro-F1 score of 77.3. Thus, in the fifth experiment, the embodiment as described herein achieved only marginally lower performance, but did so while having a several-order-of-magnitude-smaller computational footprint.

During a sixth experiment, a pre-trained LLM was fine-tuned on 200 sixth samples from the sixth verification dataset and was then executed on the remainder of the sixth verification dataset. The fine-tuned LLM in such sixth experiment achieved a macro-F1 score of 54.7. Also during that sixth experiment, an embodiment as described herein was reduced to practice using those 200 sixth samples and was then used to classify the remainder of the sixth verification dataset, achieving a macro-F1 score of 58.6. Thus, in the sixth experiment, the embodiment as described herein achieved slightly better performance, notwithstanding having a several-order-of-magnitude-smaller computational footprint.

During a seventh experiment, a pre-trained LLM was fine-tuned on 5000 seventh samples from the seventh verification dataset and was then executed on the remainder of the seventh verification dataset. The fine-tuned LLM in such seventh experiment achieved a macro-F1 score of 92.2. Also during that seventh experiment, an embodiment as described herein was reduced to practice using those 5000 seventh samples and was then used to classify the remainder of the seventh verification dataset, achieving a macro-F1 score of 90.9. Thus, in the seventh experiment, the embodiment as described herein achieved nearly comparable performance, notwithstanding having a several-order-of-magnitude-smaller computational footprint and without the hours-long fine-tuning process required for 5000 samples.

During an eighth experiment, a pre-trained LLM was fine-tuned on 5000 eighth samples from the eighth verification dataset and was then executed on the remainder of the eighth verification dataset. The fine-tuned LLM in such eighth experiment achieved a macro-F1 score of 73.0. Also during that eighth experiment, an embodiment as described herein was reduced to practice using those 5000 eighth samples and was then used to classify the remainder of the eighth verification dataset, achieving a macro-F1 score of 74.1. Thus, in the eighth experiment, the embodiment as described herein achieved comparable performance, notwithstanding having a several-order-of-magnitude-smaller computational footprint and without the hours-long fine-tuning process required for 5000 samples.

During a ninth experiment, a pre-trained LLM was fine-tuned on 5000 ninth samples from the ninth verification dataset and was then executed on the remainder of the ninth verification dataset. The fine-tuned LLM in such ninth experiment achieved a macro-F1 score of 98.3. Also during that ninth experiment, an embodiment as described herein was reduced to practice using those 5000 ninth samples and was then used to classify the remainder of the ninth verification dataset, achieving a macro-F1 score of 97.5. Thus, in the ninth experiment, the embodiment as described herein achieved comparable performance, notwithstanding having a several-order-of-magnitude-smaller computational footprint and without the hours-long fine-tuning process required for 5000 samples.

During a tenth experiment, a pre-trained LLM was fine-tuned on 5000 tenth samples from the tenth verification dataset and was then executed on the remainder of the tenth verification dataset. The fine-tuned LLM in such tenth experiment achieved a macro-F1 score of 78.8. Also during that tenth experiment, an embodiment as described herein was reduced to practice using those 5000 tenth samples and was then used to classify the remainder of the tenth verification dataset, achieving a macro-F1 score of 79.0. Thus, in the tenth experiment, the embodiment as described herein achieved comparable performance, notwithstanding having a several-order-of-magnitude-smaller computational footprint and without the hours-long fine-tuning process required for 5000 samples.

During an eleventh experiment, a pre-trained LLM was fine-tuned on 5000 eleventh samples from the eleventh verification dataset and was then executed on the remainder of the eleventh verification dataset. The fine-tuned LLM in such eleventh experiment achieved a macro-F1 score of 89.3. Also during that eleventh experiment, an embodiment as described herein was reduced to practice using those 5000 eleventh samples and was then used to classify the remainder of the eleventh verification dataset, achieving a macro-F1 score of 83.5. Thus, in the eleventh experiment, the embodiment as described herein achieved nearly comparable performance, notwithstanding having a several-order-of-magnitude-smaller computational footprint and without the hours-long fine-tuning process required for 5000 samples.

During a twelfth experiment, a pre-trained LLM was fine-tuned on 5000 twelfth samples from the twelfth verification dataset and was then executed on the remainder of the twelfth verification dataset. The fine-tuned LLM in such twelfth experiment achieved a macro-F1 score of 75.6. Also during that twelfth experiment, an embodiment as described herein was reduced to practice using those 5000 twelfth samples and was then used to classify the remainder of the twelfth verification dataset, achieving a macro-F1 score of 67.4. Thus, in the twelfth experiment, the embodiment as described herein achieved only marginally lower performance, notwithstanding having a several-order-of-magnitude-smaller computational footprint and without the hours-long fine-tuning process required for 5000 samples.

As these experimental results show, various embodiments described herein are able to provide satisfactory text classification accuracy (and, in some cases, better classification accuracy) as existing techniques, without suffering the various technical disadvantages of existing techniques (e.g., without excessive computational footprint, without needing a tailored instantiation for each site, without needing continual learning). Accordingly, these experimental results show that various embodiments described herein certainly constitute a concrete and tangible technical improvement in the field of text classification.

Although various embodiments described herein mainly pertain to classification of medical orders, it is to be appreciated that these are mere non-limiting examples for ease of explanation and illustration. In various aspects, various embodiments described herein can be extrapolated or generalized to perform computationally in-expensive classification without the hurdles of continual learning on any suitable types of texts or documents, even on non-medical texts or documents.

In various instances, machine learning algorithms or models can be implemented in any suitable way to facilitate any suitable aspects described herein. To facilitate some of the above-described machine learning aspects of various embodiments, consider the following discussion of artificial intelligence (AI). Various embodiments described herein can employ artificial intelligence to facilitate automating one or more features or functionalities. The components can employ various AI-based schemes for carrying out various embodiments/examples disclosed herein. In order to provide for or aid in the numerous determinations (e.g., determine, ascertain, infer, calculate, predict, prognose, estimate, derive, forecast, detect, compute) described herein, components described herein can examine the entirety or a subset of the data to which it is granted access and can provide for reasoning about or determine states of the system or environment from a set of observations as captured via events or data. Determinations can be employed to identify a specific context or action, or can generate a probability distribution over states, for example. The determinations can be probabilistic; that is, the computation of a probability distribution over states of interest based on a consideration of data and events. Determinations can also refer to techniques employed for composing higher-level events from a set of events or data.

Such determinations can result in the construction of new events or actions from a set of observed events or stored event data, whether or not the events are correlated in close temporal proximity, and whether the events and data come from one or several event and data sources. Components disclosed herein can employ various classification (explicitly trained (e.g., via training data) as well as implicitly trained (e.g., via observing behavior, preferences, historical information, receiving extrinsic information, and so on)) schemes or systems (e.g., support vector machines, neural networks, expert systems, Bayesian belief networks, fuzzy logic, data fusion engines, and so on) in connection with performing automatic or determined action in connection with the claimed subject matter. Thus, classification schemes or systems can be used to automatically learn and perform a number of functions, actions, or determinations.

A classifier can map an input attribute vector, z=(z₁, z₂, z₃, z₄, zₙ), to a confidence that the input belongs to a class, as by f(z)=confidence (class). Such classification can employ a probabilistic or statistical-based analysis (e.g., factoring into the analysis utilities and costs) to determinate an action to be automatically performed. A support vector machine (SVM) can be an example of a classifier that can be employed. The SVM operates by finding a hyper-surface in the space of possible inputs, where the hyper-surface attempts to split the triggering criteria from the non-triggering events. Intuitively, this makes the classification correct for testing data that is near, but not identical to training data. Other directed and undirected model classification approaches include, e.g., naïve Bayes, Bayesian networks, decision trees, neural networks, fuzzy logic models, or probabilistic classification models providing different patterns of independence, any of which can be employed. Classification as used herein also is inclusive of statistical regression that is utilized to develop models of priority.

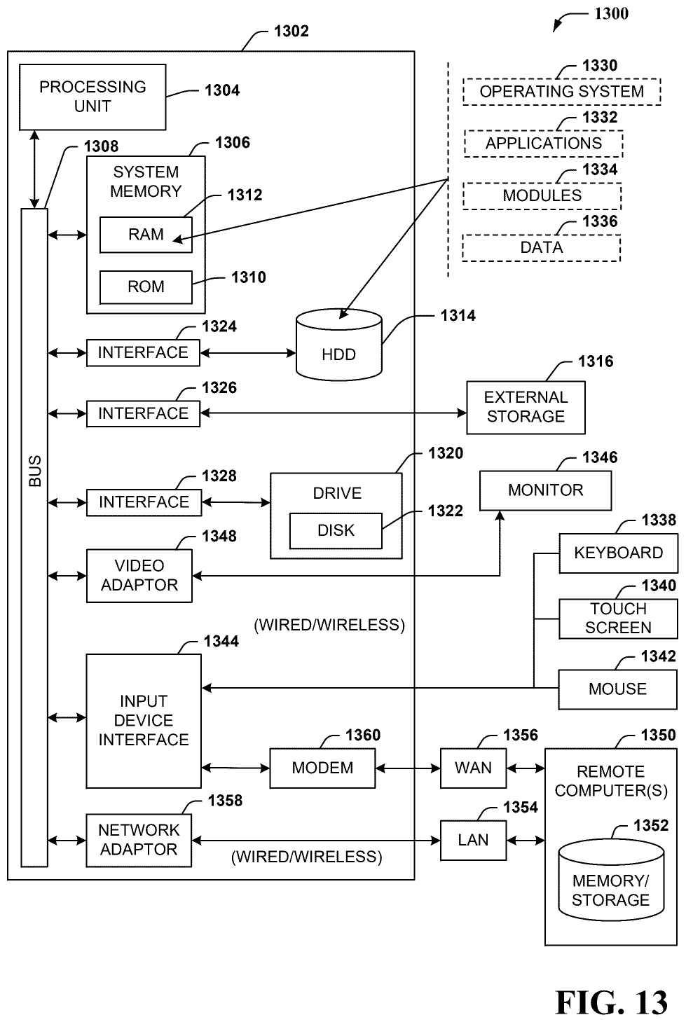

In order to provide additional context for various embodiments described herein, FIG. 13 and the following discussion are intended to provide a brief, general description of a suitable computing environment 1300 in which the various embodiments of the embodiment described herein can be implemented. While the embodiments have been described above in the general context of computer-executable instructions that can run on one or more computers, those skilled in the art will recognize that the embodiments can be also implemented in combination with other program modules or as a combination of hardware and software.

Generally, program modules include routines, programs, components, data structures, etc., that perform particular tasks or implement particular abstract data types. Moreover, those skilled in the art will appreciate that the inventive methods can be practiced with other computer system configurations, including single-processor or multi-processor computer systems, minicomputers, mainframe computers, Internet of Things (IoT) devices, distributed computing systems, as well as personal computers, hand-held computing devices, microprocessor-based or programmable consumer electronics, and the like, each of which can be operatively coupled to one or more associated devices.

The illustrated embodiments of the embodiments herein can be also practiced in distributed computing environments where certain tasks are performed by remote processing devices that are linked through a communications network. In a distributed computing environment, program modules can be located in both local and remote memory storage devices.

Computing devices typically include a variety of media, which can include computer-readable storage media, machine-readable storage media, or communications media, which two terms are used herein differently from one another as follows. Computer-readable storage media or machine-readable storage media can be any available storage media that can be accessed by the computer and includes both volatile and nonvolatile media, removable and non-removable media. By way of example, and not limitation, computer-readable storage media or machine-readable storage media can be implemented in connection with any method or technology for storage of information such as computer-readable or machine-readable instructions, program modules, structured data or unstructured data.

Computer-readable storage media can include, but are not limited to, random access memory (RAM), read only memory (ROM), electrically erasable programmable read only memory (EEPROM), flash memory or other memory technology, compact disk read only memory (CD-ROM), digital versatile disk (DVD), Blu-ray disc (BD) or other optical disk storage, magnetic cassettes, magnetic tape, magnetic disk storage or other magnetic storage devices, solid state drives or other solid state storage devices, or other tangible or non-transitory media which can be used to store desired information. In this regard, the terms “tangible” or “non-transitory” herein as applied to storage, memory or computer-readable media, are to be understood to exclude only propagating transitory signals per se as modifiers and do not relinquish rights to all standard storage, memory or computer-readable media that are not only propagating transitory signals per se.

Computer-readable storage media can be accessed by one or more local or remote computing devices, e.g., via access requests, queries or other data retrieval protocols, for a variety of operations with respect to the information stored by the medium.

Communications media typically embody computer-readable instructions, data structures, program modules or other structured or unstructured data in a data signal such as a modulated data signal, e.g., a carrier wave or other transport mechanism, and includes any information delivery or transport media. The term “modulated data signal” or signals refers to a signal that has one or more of its characteristics set or changed in such a manner as to encode information in one or more signals. By way of example, and not limitation, communication media include wired media, such as a wired network or direct-wired connection, and wireless media such as acoustic, RF, infrared and other wireless media.

With reference again to FIG. 13, the example environment 1300 for implementing various embodiments of the aspects described herein includes a computer 1302, the computer 1302 including a processing unit 1304, a system memory 1306 and a system bus 1308. The system bus 1308 couples system components including, but not limited to, the system memory 1306 to the processing unit 1304. The processing unit 1304 can be any of various commercially available processors. Dual microprocessors and other multi-processor architectures can also be employed as the processing unit 1304.

The system bus 1308 can be any of several types of bus structure that can further interconnect to a memory bus (with or without a memory controller), a peripheral bus, and a local bus using any of a variety of commercially available bus architectures. The system memory 1306 includes ROM 1310 and RAM 1312. A basic input/output system (BIOS) can be stored in a non-volatile memory such as ROM, erasable programmable read only memory (EPROM), EEPROM, which BIOS contains the basic routines that help to transfer information between elements within the computer 1302, such as during startup. The RAM 1312 can also include a high-speed RAM such as static RAM for caching data.

The computer 1302 further includes an internal hard disk drive (HDD) 1314 (e.g., EIDE, SATA), one or more external storage devices 1316 (e.g., a magnetic floppy disk drive (FDD) 1316, a memory stick or flash drive reader, a memory card reader, etc.) and a drive 1320, e.g., such as a solid state drive, an optical disk drive, which can read or write from a disk 1322, such as a CD-ROM disc, a DVD, a BD, etc. Alternatively, where a solid state drive is involved, disk 1322 would not be included, unless separate. While the internal HDD 1314 is illustrated as located within the computer 1302, the internal HDD 1314 can also be configured for external use in a suitable chassis (not shown). Additionally, while not shown in environment 1300, a solid state drive (SSD) could be used in addition to, or in place of, an HDD 1314. The HDD 1314, external storage device(s) 1316 and drive 1320 can be connected to the system bus 1308 by an HDD interface 1324, an external storage interface 1326 and a drive interface 1328, respectively. The interface 1324 for external drive implementations can include at least one or both of Universal Serial Bus (USB) and Institute of Electrical and Electronics Engineers (IEEE) 1394 interface technologies. Other external drive connection technologies are within contemplation of the embodiments described herein.

The drives and their associated computer-readable storage media provide nonvolatile storage of data, data structures, computer-executable instructions, and so forth. For the computer 1302, the drives and storage media accommodate the storage of any data in a suitable digital format. Although the description of computer-readable storage media above refers to respective types of storage devices, it should be appreciated by those skilled in the art that other types of storage media which are readable by a computer, whether presently existing or developed in the future, could also be used in the example operating environment, and further, that any such storage media can contain computer-executable instructions for performing the methods described herein.

A number of program modules can be stored in the drives and RAM 1312, including an operating system 1330, one or more application programs 1332, other program modules 1334 and program data 1336. All or portions of the operating system, applications, modules, or data can also be cached in the RAM 1312. The systems and methods described herein can be implemented utilizing various commercially available operating systems or combinations of operating systems.

Computer 1302 can optionally comprise emulation technologies. For example, a hypervisor (not shown) or other intermediary can emulate a hardware environment for operating system 1330, and the emulated hardware can optionally be different from the hardware illustrated in FIG. 13. In such an embodiment, operating system 1330 can comprise one virtual machine (VM) of multiple VMs hosted at computer 1302. Furthermore, operating system 1330 can provide runtime environments, such as the Java runtime environment or the .NET framework, for applications 1332. Runtime environments are consistent execution environments that allow applications 1332 to run on any operating system that includes the runtime environment. Similarly, operating system 1330 can support containers, and applications 1332 can be in the form of containers, which are lightweight, standalone, executable packages of software that include, e.g., code, runtime, system tools, system libraries and settings for an application.

Further, computer 1302 can be enable with a security module, such as a trusted processing module (TPM). For instance with a TPM, boot components hash next in time boot components, and wait for a match of results to secured values, before loading a next boot component. This process can take place at any layer in the code execution stack of computer 1302, e.g., applied at the application execution level or at the operating system (OS) kernel level, thereby enabling security at any level of code execution.

A user can enter commands and information into the computer 1302 through one or more wired/wireless input devices, e.g., a keyboard 1338, a touch screen 1340, and a pointing device, such as a mouse 1342. Other input devices (not shown) can include a microphone, an infrared (IR) remote control, a radio frequency (RF) remote control, or other remote control, a joystick, a virtual reality controller or virtual reality headset, a game pad, a stylus pen, an image input device, e.g., camera(s), a gesture sensor input device, a vision movement sensor input device, an emotion or facial detection device, a biometric input device, e.g., fingerprint or iris scanner, or the like. These and other input devices are often connected to the processing unit 1304 through an input device interface 1344 that can be coupled to the system bus 1308, but can be connected by other interfaces, such as a parallel port, an IEEE 1394 serial port, a game port, a USB port, an IR interface, a BLUETOOTH® interface, etc.

A monitor 1346 or other type of display device can be also connected to the system bus 1308 via an interface, such as a video adapter 1348. In addition to the monitor 1346, a computer typically includes other peripheral output devices (not shown), such as speakers, printers, etc.

The computer 1302 can operate in a networked environment using logical connections via wired or wireless communications to one or more remote computers, such as a remote computer(s) 1350. The remote computer(s) 1350 can be a workstation, a server computer, a router, a personal computer, portable computer, microprocessor-based entertainment appliance, a peer device or other common network node, and typically includes many or all of the elements described relative to the computer 1302, although, for purposes of brevity, only a memory/storage device 1352 is illustrated. The logical connections depicted include wired/wireless connectivity to a local area network (LAN) 1354 or larger networks, e.g., a wide area network (WAN) 1356. Such LAN and WAN networking environments are commonplace in offices and companies, and facilitate enterprise-wide computer networks, such as intranets, all of which can connect to a global communications network, e.g., the Internet.

When used in a LAN networking environment, the computer 1302 can be connected to the local network 1354 through a wired or wireless communication network interface or adapter 1358. The adapter 1358 can facilitate wired or wireless communication to the LAN 1354, which can also include a wireless access point (AP) disposed thereon for communicating with the adapter 1358 in a wireless mode.

When used in a WAN networking environment, the computer 1302 can include a modem 1360 or can be connected to a communications server on the WAN 1356 via other means for establishing communications over the WAN 1356, such as by way of the Internet. The modem 1360, which can be internal or external and a wired or wireless device, can be connected to the system bus 1308 via the input device interface 1344. In a networked environment, program modules depicted relative to the computer 1302 or portions thereof, can be stored in the remote memory/storage device 1352. It will be appreciated that the network connections shown are example and other means of establishing a communications link between the computers can be used.

When used in either a LAN or WAN networking environment, the computer 1302 can access cloud storage systems or other network-based storage systems in addition to, or in place of, external storage devices 1316 as described above, such as but not limited to a network virtual machine providing one or more aspects of storage or processing of information. Generally, a connection between the computer 1302 and a cloud storage system can be established over a LAN 1354 or WAN 1356 e.g., by the adapter 1358 or modem 1360, respectively. Upon connecting the computer 1302 to an associated cloud storage system, the external storage interface 1326 can, with the aid of the adapter 1358 or modem 1360, manage storage provided by the cloud storage system as it would other types of external storage. For instance, the external storage interface 1326 can be configured to provide access to cloud storage sources as if those sources were physically connected to the computer 1302.

The computer 1302 can be operable to communicate with any wireless devices or entities operatively disposed in wireless communication, e.g., a printer, scanner, desktop or portable computer, portable data assistant, communications satellite, any piece of equipment or location associated with a wirelessly detectable tag (e.g., a kiosk, news stand, store shelf, etc.), and telephone. This can include Wireless Fidelity (Wi-Fi) and BLUETOOTH® wireless technologies. Thus, the communication can be a predefined structure as with a conventional network or simply an ad hoc communication between at least two devices.

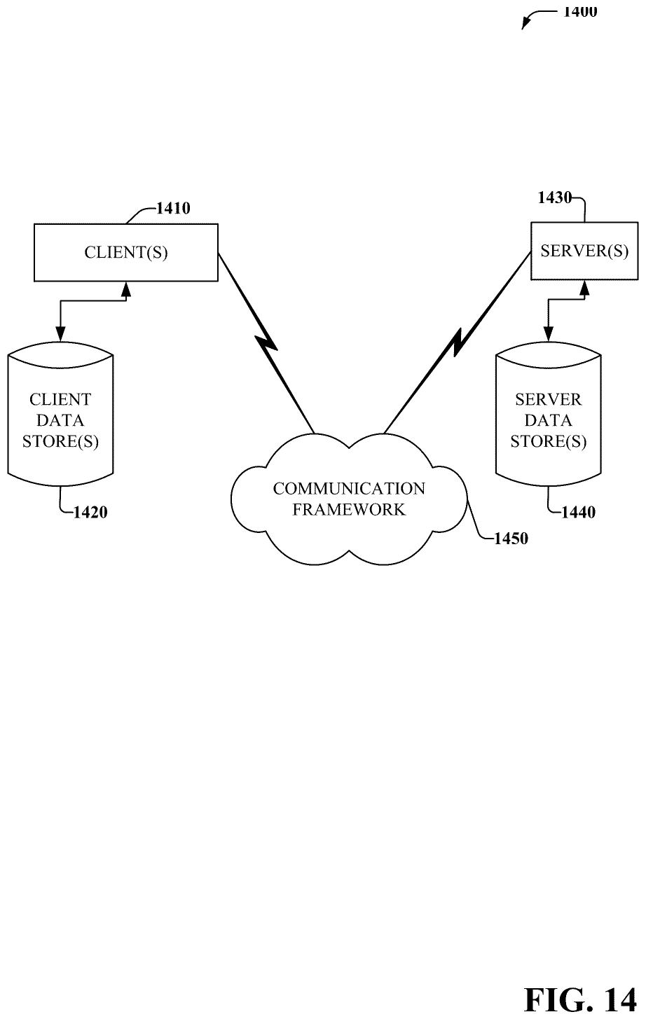

FIG. 14 is a schematic block diagram of a sample computing environment 1400 with which the disclosed subject matter can interact. The sample computing environment 1400 includes one or more client(s) 1410. The client(s) 1410 can be hardware or software (e.g., threads, processes, computing devices). The sample computing environment 1400 also includes one or more server(s) 1430. The server(s) 1430 can also be hardware or software (e.g., threads, processes, computing devices). The servers 1430 can house threads to perform transformations by employing one or more embodiments as described herein, for example. One possible communication between a client 1410 and a server 1430 can be in the form of a data packet adapted to be transmitted between two or more computer processes. The sample computing environment 1400 includes a communication framework 1450 that can be employed to facilitate communications between the client(s) 1410 and the server(s) 1430. The client(s) 1410 are operably connected to one or more client data store(s) 1420 that can be employed to store information local to the client(s) 1410. Similarly, the server(s) 1430 are operably connected to one or more server data store(s) 1440 that can be employed to store information local to the servers 1430.

Various embodiments may be a system, a method, an apparatus or a computer program product at any possible technical detail level of integration. The computer program product can include a computer readable storage medium (or media) having computer readable program instructions thereon for causing a processor to carry out aspects of various embodiments. The computer readable storage medium can be a tangible device that can retain and store instructions for use by an instruction execution device. The computer readable storage medium can be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium can also include the following: a portable computer diskette, a hard disk, a solid state drive such as M.2 (including non-volatile memory express (NVMe) or serial advanced technology attachment (SATA)), a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network or a wireless network. The network can comprise copper transmission cables, optical transmission fibers, wireless transmission, routers, firewalls, switches, gateway computers or edge servers. A network adapter card or network interface in each computing/processing device receives computer readable program instructions from the network and forwards the computer readable program instructions for storage in a computer readable storage medium within the respective computing/processing device. Computer readable program instructions for carrying out operations of various embodiments can be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions can execute entirely on the user's computer, partly on the user's computer, as a stand-alone software package, partly on the user's computer and partly on a remote computer or entirely on the remote computer or server. In the latter scenario, the remote computer can be connected to the user's computer through any type of network, including a local area network (LAN) or a wide area network (WAN), or the connection can be made to an external computer (for example, through the Internet using an Internet Service Provider). In some embodiments, electronic circuitry including, for example, programmable logic circuitry, field-programmable gate arrays (FPGA), or programmable logic arrays (PLA) can execute the computer readable program instructions by utilizing state information of the computer readable program instructions to personalize the electronic circuitry, in order to perform various aspects.

Various aspects are described herein with reference to flowchart illustrations or block diagrams of methods, apparatus (systems), and computer program products according to various embodiments. It will be understood that each block of the flowchart illustrations or block diagrams, and combinations of blocks in the flowchart illustrations or block diagrams, can be implemented by computer readable program instructions. These computer readable program instructions can be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart or block diagram block or blocks. These computer readable program instructions can also be stored in a computer readable storage medium that can direct a computer, a programmable data processing apparatus, or other devices to function in a particular manner, such that the computer readable storage medium having instructions stored therein comprises an article of manufacture including instructions which implement aspects of the function/act specified in the flowchart or block diagram block or blocks. The computer readable program instructions can also be loaded onto a computer, other programmable data processing apparatus, or other device to cause a series of operational acts to be performed on the computer, other programmable apparatus or other device to produce a computer implemented process, such that the instructions which execute on the computer, other programmable apparatus, or other device implement the functions/acts specified in the flowchart or block diagram block or blocks.

The flowcharts and block diagrams in the Figures illustrate the architecture, functionality, and operation of possible implementations of systems, methods, and computer program products according to various embodiments. In this regard, each block in the flowchart or block diagrams can represent a module, segment, or portion of instructions, which comprises one or more executable instructions for implementing the specified logical function(s). In some alternative implementations, the functions noted in the blocks can occur out of the order noted in the Figures. For example, two blocks shown in succession can, in fact, be executed substantially concurrently, or the blocks can sometimes be executed in the reverse order, depending upon the functionality involved. It will also be noted that each block of the block diagrams or flowchart illustration, and combinations of blocks in the block diagrams or flowchart illustration, can be implemented by special purpose hardware-based systems that perform the specified functions or acts or carry out combinations of special purpose hardware and computer instructions.

While the subject matter has been described above in the general context of computer-executable instructions of a computer program product that runs on a computer or computers, those skilled in the art will recognize that this disclosure also can or can be implemented in combination with other program modules. Generally, program modules include routines, programs, components, data structures, etc. that perform particular tasks or implement particular abstract data types. Moreover, those skilled in the art will appreciate that various aspects can be practiced with other computer system configurations, including single-processor or multiprocessor computer systems, mini-computing devices, mainframe computers, as well as computers, hand-held computing devices (e.g., PDA, phone), microprocessor-based or programmable consumer or industrial electronics, and the like. The illustrated aspects can also be practiced in distributed computing environments in which tasks are performed by remote processing devices that are linked through a communications network. However, some, if not all aspects of this disclosure can be practiced on stand-alone computers. In a distributed computing environment, program modules can be located in both local and remote memory storage devices.

As used in this application, the terms “component,” “system,” “platform,” “interface,” and the like, can refer to or can include a computer-related entity or an entity related to an operational machine with one or more specific functionalities. The entities disclosed herein can be either hardware, a combination of hardware and software, software, or software in execution. For example, a component can be, but is not limited to being, a process running on a processor, a processor, an object, an executable, a thread of execution, a program, or a computer. By way of illustration, both an application running on a server and the server can be a component. One or more components can reside within a process or thread of execution and a component can be localized on one computer or distributed between two or more computers. In another example, respective components can execute from various computer readable media having various data structures stored thereon. The components can communicate via local or remote processes such as in accordance with a signal having one or more data packets (e.g., data from one component interacting with another component in a local system, distributed system, or across a network such as the Internet with other systems via the signal). As another example, a component can be an apparatus with specific functionality provided by mechanical parts operated by electric or electronic circuitry, which is operated by a software or firmware application executed by a processor. In such a case, the processor can be internal or external to the apparatus and can execute at least a part of the software or firmware application. As yet another example, a component can be an apparatus that provides specific functionality through electronic components without mechanical parts, wherein the electronic components can include a processor or other means to execute software or firmware that confers at least in part the functionality of the electronic components. In an aspect, a component can emulate an electronic component via a virtual machine, e.g., within a cloud computing system.

In addition, the term “or” is intended to mean an inclusive “or” rather than an exclusive “or.” That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances. As used herein, the term “and/or” is intended to have the same meaning as “or.” Moreover, articles “a” and “an” as used in the subject specification and annexed drawings should generally be construed to mean “one or more” unless specified otherwise or clear from context to be directed to a singular form. As used herein, the terms “example” or “exemplary” are utilized to mean serving as an example, instance, or illustration. For the avoidance of doubt, the subject matter disclosed herein is not limited by such examples. In addition, any aspect or design described herein as an “example” or “exemplary” is not necessarily to be construed as preferred or advantageous over other aspects or designs, nor is it meant to preclude equivalent exemplary structures and techniques known to those of ordinary skill in the art.

The herein disclosure describes non-limiting examples. For ease of description or explanation, various portions of the herein disclosure utilize the term “each,” “every,” or “all” when discussing various examples. Such usages of the term “each,” “every,” or “all” are non-limiting. In other words, when the herein disclosure provides a description that is applied to “each,” “every,” or “all” of some particular object or component, it should be understood that this is a non-limiting example, and it should be further understood that, in various other examples, it can be the case that such description applies to fewer than “each,” “every,” or “all” of that particular object or component.

As it is employed in the subject specification, the term “processor” can refer to substantially any computing processing unit or device comprising, but not limited to, single-core processors; single-processors with software multithread execution capability; multi-core processors; multi-core processors with software multithread execution capability; multi-core processors with hardware multithread technology; parallel platforms; and parallel platforms with distributed shared memory. Additionally, a processor can refer to an integrated circuit, an application specific integrated circuit (ASIC), a digital signal processor (DSP), a field programmable gate array (FPGA), a programmable logic controller (PLC), a complex programmable logic device (CPLD), a discrete gate or transistor logic, discrete hardware components, or any combination thereof designed to perform the functions described herein. Further, processors can exploit nano-scale architectures such as, but not limited to, molecular and quantum-dot based transistors, switches and gates, in order to optimize space usage or enhance performance of user equipment. A processor can also be implemented as a combination of computing processing units. In this disclosure, terms such as “store,” “storage,” “data store,” data storage,” “database,” and substantially any other information storage component relevant to operation and functionality of a component are utilized to refer to “memory components,” entities embodied in a “memory,” or components comprising a memory. It is to be appreciated that memory or memory components described herein can be either volatile memory or nonvolatile memory, or can include both volatile and nonvolatile memory. By way of illustration, and not limitation, nonvolatile memory can include read only memory (ROM), programmable ROM (PROM), electrically programmable ROM (EPROM), electrically erasable ROM (EEPROM), flash memory, or nonvolatile random access memory (RAM) (e.g., ferroelectric RAM (FeRAM). Volatile memory can include RAM, which can act as external cache memory, for example. By way of illustration and not limitation, RAM is available in many forms such as synchronous RAM (SRAM), dynamic RAM (DRAM), synchronous DRAM (SDRAM), double data rate SDRAM (DDR SDRAM), enhanced SDRAM (ESDRAM), Synchlink DRAM (SLDRAM), direct Rambus RAM (DRRAM), direct Rambus dynamic RAM (DRDRAM), and Rambus dynamic RAM (RDRAM). Additionally, the disclosed memory components of systems or computer-implemented methods herein are intended to include, without being limited to including, these and any other suitable types of memory.

What has been described above include mere examples of systems and computer-implemented methods. It is, of course, not possible to describe every conceivable combination of components or computer-implemented methods for purposes of describing this disclosure, but many further combinations and permutations of this disclosure are possible. Furthermore, to the extent that the terms “includes,” “has,” “possesses,” and the like are used in the detailed description, claims, appendices and drawings such terms are intended to be inclusive in a manner similar to the term “comprising” as “comprising” is interpreted when employed as a transitional word in a claim.

The descriptions of the various embodiments have been presented for purposes of illustration, but are not intended to be exhaustive or limited to the embodiments disclosed. Many modifications and variations will be apparent without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A system, comprising:
a processor that executes computer-executable components stored in a non-transitory computer-readable memory, wherein the computer-executable components comprise:
an access component that accesses a new medical order associated with a medical patient;
a vector component that computes:
one or more global vector representations of the new medical order; and
one or more local vector representations for respective ones or combinations of a set of textual sections that make up the new medical order, thereby yielding a set of local vector representations of the new medical order; and
a search component that identifies a new classification label for the new medical order, based on searching an historical order-label database using both the set of global vector representations and the set of local vector representations.

2. The system of claim 1, wherein the vector component generates the one or more global vector representations and the set of local vector representations via a term-frequency-inverse-domain-frequency vectorizer or via one or more encoders of one or more respective, pre-trained large language models.

3. The system of claim 1, wherein:
the historical order-label database comprises:
a plurality of past medical orders;
a plurality of past classification labels respectively corresponding to the plurality of past medical orders;
one or more past global vector representations for each respective one of the plurality of past medical orders; and
a set of past local vector representations for each respective one of the plurality of past medical orders; and
the search component:
compares the one or more global vector representations of the new medical order to the one or more past global vector representations of each respective one of the plurality of past medical orders, thereby yielding one or more global similarity scores or ranks for each respective one of the plurality of past medical orders; and
compares the set of local vector representations of the new medical order to the set of past local vector representations of each respective one of the plurality of past medical orders, thereby yielding a set of local similarity scores or ranks for each respective one of the plurality of past medical orders.

4. The system of claim 3, wherein the search component:
aggregates the one or more global similarity scores or ranks and the set of local similarity scores or ranks for each respective one of the plurality of past medical orders, thereby yielding an aggregate similarity score or rank for each respective one of the plurality of past medical orders; and
identifies, from the plurality of past medical orders and based on the aggregate similarity scores or ranks, a most-similar past medical order, wherein the new classification label is whichever of the plurality of past classification labels that corresponds to the most-similar past medical order.

5. The system of claim 4, wherein the search component inserts the new medical order, the one or more global vector representations, the set of local vector representations, and the new classification label as a new entry into the historical order-label database.

6. The system of claim 1, wherein the medical patient is associated with a medical imaging scanner, wherein the new classification label specifies an imaging protocol for the medical imaging scanner, and wherein the computer-executable components comprise:
an execution component that causes the medical imaging scanner to scan the medical patient according to the imaging protocol.

7. The system of claim 1, wherein an airway or blood vessel of the medical patient is coupled to a tank containing a fluidic medication, wherein the new classification label specifies a dosage, and wherein the computer-executable components comprise:
an execution component that causes a pump of the tank to dispense the fluidic medication to the airway or blood vessel of the medical patient in accordance with the dosage.

8. The system of claim 1, wherein the medical patient is associated with a robotic surgery apparatus, wherein the new classification label specifies a surgical intervention, and wherein the computer-executable components comprise:
an execution component that causes the robotic surgery apparatus to perform the surgical intervention on the medical patient.

9. A computer-implemented method, comprising:
accessing, by a device operatively coupled to a processor, a new medical order associated with a medical patient;
computing, by the device:
one or more global vector representations of the new medical order; and
one or more local vector representations for respective ones or combinations of a set of textual sections that make up the new medical order, thereby yielding a set of local vector representations of the new medical order; and
identifying, by the device, a new classification label for the new medical order, based on searching an historical order-label database using both the set of global vector representations and the set of local vector representations.

10. The computer-implemented method of claim 9, wherein the device generates the one or more global vector representations and the set of local vector representations via a term-frequency-inverse-domain-frequency vectorizer or via one or more encoders of one or more respective, pre-trained large language models.

11. The computer-implemented method of claim 9, wherein:
the historical order-label database comprises:
a plurality of past medical orders;
a plurality of past classification labels respectively corresponding to the plurality of past medical orders;
one or more past global vector representations for each respective one of the plurality of past medical orders; and
a set of past local vector representations for each respective one of the plurality of past medical orders; and further comprising:
comparing, by the device, the one or more global vector representations of the new medical order to the one or more past global vector representations of each respective one of the plurality of past medical orders, thereby yielding one or more global similarity scores or ranks for each respective one of the plurality of past medical orders; and
comparing, by the device, the set of local vector representations of the new medical order to the set of past local vector representations of each respective one of the plurality of past medical orders, thereby yielding a set of local similarity scores or ranks for each respective one of the plurality of past medical orders.

12. The computer-implemented method of claim 11, further comprising:
aggregating, by the device, the one or more global similarity scores or ranks and the set of local similarity scores or ranks for each respective one of the plurality of past medical orders, thereby yielding an aggregate similarity score or rank for each respective one of the plurality of past medical orders; and
identifying, by the device, from the plurality of past medical orders, and based on the aggregate similarity scores or ranks, a most-similar past medical order, wherein the new classification label is whichever of the plurality of past classification labels that corresponds to the most-similar past medical order.

13. The computer-implemented method of claim 12, further comprising:
inserting, by the device, the new medical order, the one or more global vector representations, the set of local vector representations, and the new classification label as a new entry into the historical order-label database.

14. The computer-implemented method of claim 9, wherein the medical patient is associated with a medical imaging scanner, wherein the new classification label specifies an imaging protocol for the medical imaging scanner, and further comprising:
causing, by the device, the medical imaging scanner to scan the medical patient according to the imaging protocol.

15. The computer-implemented method of claim 9, wherein an airway or blood vessel of the medical patient is coupled to a tank containing a fluidic medication, wherein the new classification label specifies a dosage, and further comprising:
causing, by the device, a pump of the tank to dispense the fluidic medication to the airway or blood vessel of the medical patient in accordance with the dosage.

16. The computer-implemented method of claim 9, wherein the medical patient is associated with a robotic surgery apparatus, wherein the new classification label specifies a surgical intervention, and further comprising:
causing, by the device, the robotic surgery apparatus to perform the surgical intervention on the medical patient.

17. A computer program product for facilitating global and local search-based classification of text, the computer program product comprising a non-transitory computer-readable memory having program instructions embodied therewith, the program instructions executable by a processor to cause the processor to:
access a new textual document;
compute:
one or more global vector representations of the new textual document; and
one or more local vector representations for respective ones or combinations of a set of sections of the new textual document, thereby yielding a set of local vector representations of the new textual document; and
identify a new classification label for the new textual document, based on searching an historical document-label database using both the set of global vector representations and the set of local vector representations.

18. The computer program product of claim 17, wherein:
the historical document-label database comprises:
a plurality of past textual documents respectively corresponding to a plurality of past classification labels;
one or more past global vector representations for each respective one of the plurality of past textual documents; and
a set of past local vector representations for each respective one of the plurality of past textual documents; and
the program instructions are further executable to cause the processor to:
compare the one or more global vector representations of the new textual document to the one or more past global vector representations of each respective one of the plurality of past textual documents, thereby yielding one or more global similarity scores or ranks for each respective one of the plurality of past textual documents; and
compare the set of local vector representations of the new textual document to the set of past local vector representations of each respective one of the plurality of past textual documents, thereby yielding a set of local similarity scores or ranks for each respective one of the plurality of past textual documents.

19. The computer program product of claim 18, wherein the program instructions are further executable to cause the processor to:
aggregate the one or more global similarity scores or ranks and the set of local similarity scores or ranks for each respective one of the plurality of past textual documents, thereby yielding an aggregate similarity score or rank for each respective one of the plurality of past textual documents; and
identify, from the plurality of past medical orders and based on the average similarity scores or ranks, a most-similar past textual document, wherein the new classification label is whichever of the plurality of past classification labels that corresponds to the most-similar past textual document.

20. The computer program product of claim 19, wherein the program instructions are further executable to cause the processor to:
insert the new textual document, the one or more global vector representations, the set of local vector representations, and the new classification label into the historical document-label database.
